(19) United States
(12) Patent Application Publication
(10) Pub. No.: US 2024/0386891 A1
(43) Pub. Date: Nov. 21, 2024

(54) INFORMATION PROCESSING APPARATUS, INFORMATION PROCESSING METHOD, AND PROGRAM

(71) Applicant: Sony Group Corporation, Tokyo (JP)

(72) Inventors: Shinichi KAWANO, Tokyo (JP); Naoki INOUE, Oita (JP); Yuki KAWANO, Oita (JP); Hiro IWASE, Kanagawa (JP); Takayoshi YAMASAKI, Tokyo (JP)

(73) Assignee: Sony Group Corporation, Tokyo (JP)

(21) Appl. No.: 18/692,829

(22) PCT Filed: Sep. 21, 2022
(86) PCT No.: PCT/JP2022/035060

§ 371 Date: Mar. 18, 2024

(30) Foreign Application Priority Data
Oct. 4, 2021 (JP) 2021-163657

(51) Int. Cl. G10L 15/22; G06F 3/01; G10L 15/25; G10L 15/26

(52) U.S. Cl. CPC G10L 15/22; G06F 3/013; G10L 15/25; G10L 15/26

(57) ABSTRACT

An information processing apparatus according to an embodiment of the present technology includes an acquisition section, a determination section and a control section. The acquisition section acquires character information obtained by converting utterance of a speaker into characters by speech recognition. The determination section determines presence or absence of a conveyance intention that the speaker tries to convey speaker's own utterance content to a receiver using the character information based on a state of the speaker. The control section executes a process of displaying the character information on display devices used by respective of the speaker and the receiver, and a process of presenting a determination result regarding the conveyance intention to at least one of the speaker and the receiver.

### TECHNICAL FIELD

The present technology relates to an information processing apparatus, an information processing method, and a program that can be applied to a communication tool or the like using speech recognition.

### BACKGROUND ART

In the related art, a technique for supporting communication by displaying utterance content as characters using speech recognition has been developed. For example, Patent Literature 1 discloses a system that supports the communication by mutually displaying translation results using the speech recognition. In this system, a speech of one user is acquired by the speech recognition, and characters acquired by translating the content are displayed to the other user. In such a system, for example, if a large amount of translation results are presented, reading or the like on a receiver side may not catch up. For this reason, according to Patent Literature 1, a notification is given to a utterer side so as to temporarily stop utterance depending on a situation of the receiver side (Patent Literature 1, paragraphs [0084], [0143], [0144], [0164], FIG. 28, and the like).

### CITATION LIST

### Patent Literature

Patent Literature 1: PCT International Publication No. 2017/191713

### DISCLOSURE OF INVENTION

### Technical Problem

In this way, if communication is performed through characters acquired by the speech recognition, the communication may be hindered depending on how the tool is used. For this reason, there is a demand for a technique for realizing smooth communication using the speech recognition.

In view of the above-described circumstances, an object of the present technology is to provide an information processing apparatus, an information processing method, and a program capable of realizing the smooth communication using the speech recognition.

### Solution to Problem

In order to achieve the object described above, an information processing apparatus according to an embodiment of the present technology includes an acquisition section, a determination section and a control section.

The acquisition section acquires character information obtained by converting utterance of a speaker into characters by speech recognition.

The determination section determines presence or absence of a conveyance intention that the speaker tries to convey speaker's own utterance content to a receiver using the character information based on a state of the speaker.

The control section executes a process of displaying the character information on display devices used by respective of the speaker and the receiver, and a process of presenting a determination result regarding the conveyance intention to at least one of the speaker and the receiver.

According to the information processing apparatus, the utterance of the speaker is converted into characters by the speech recognition, and is displayed as character information on both the speaker and the receiver. At this time, based on the state of the speaker, it determines whether or not the conveyance intention is present that the speaker tries to convey the utterance content to the receiver using the character information, and the determination result is presented to the speaker or the receiver. Thus, for example, it is possible to encourage the speaker to utter while confirming the character information, or to convey the receiver to information such as whether or not to pay attention to the character information. As a result, smooth communication using the speech recognition can be realized.

The control section may generate notification data for notifying at least one of the speaker and the receiver that the conveyance intention is absent if it determines that the conveyance intention is absent.

The notification data may include at least one of visual data, haptic data, and sound data.

The information processing apparatus may further include a line-of-sight detection section that detects a line-of-sight of the speaker, and a line-of-sight determination section that determines whether or not the line-of-sight of the speaker is deviated from an area in which the character information is displayed in the display device used by the speaker based on a detection result of the line-of-sight of the speaker. In this case, the determination section may start a determination process of the conveyance intention if the line-of-sight of the speaker deviates from the area in which the character information is displayed.

The determination section may execute the determination process of the conveyance intention based on at least one of the line-of-sight of the speaker, a speech speed of the speaker, a volume of the speaker, a head direction of the speaker, or a hand position of the speaker.

The determination section may determine that the conveyance intention is absent if a state that the line-of-sight of the speaker is deviated from the area in which the character information is displayed continues for a predetermined time.

The determination section may execute the determination process of the conveyance intention based on the line-of-sight of the speaker and a line-of-sight of the receiver.

The control section may execute a process of making a field of view of the speaker difficult to look if the line-of-sight of the speaker deviates from the area in which the character information is displayed.

The control section may set a speed at which the field of view of the speaker is difficult to look based on at least one of reliability of the speech recognition, the speech speed of the speaker, a motion tendency of the line-of-sight of the speaker, or a noise level around the speaker.

The display device used by the speaker may be a transmissive display device. In this case, the control section may execute at least one of a process of decreasing transparency of at least a part of the transmissive display device or a process of displaying an object that blocks the field of view of the speaker on the transmissive display device, as a process of making the field of view of the speaker difficult to look.

The control section may cancel the process of making the field of view of the speaker difficult to look if the line-of-sight of the speaker returns to the area in which the character information is displayed.

The control section may display the character information so as to intersect the line-of-sight of the speaker in the display device used by the speaker if it determines that the conveyance intention is absent.

The control section may execute a suppression process regarding the speech recognition if it determines that the conveyance intention is absent.

The control section may stop a speech recognition process or may stop the process of displaying the character information on at least one of the display devices used by the respective of the speaker and the receiver, as the suppression process.

The control section may present to at least the receiver that the conveyance intention is present if it determines that the conveyance intention is present.

The information processing apparatus may further include a dummy information generation section that generates dummy information that the speaker appears to be uttering even if a speech of the speaker is absent. In this case, the control section may display the dummy information on the display device used by the receiver until the character information indicating the utterance content of the speaker is acquired by the speech recognition during a period in which it determines that the conveyance intention is present.

The dummy information may include at least one of information of a dummy effect that the speaker appears to be uttering or information of a dummy character string that the character information appears to be outputted.

An information processing method according to an embodiment of the present technology is an information processing method executed by a computer system, and includes acquiring character information obtained by converting utterance of a speaker into characters by speech recognition.

Based on the state of the speaker, it determines presence or absence of a conveyance intention that the speaker tries to convey speaker's own utterance content to a receiver using the character information.

A process of displaying the character information on display devices used by respective of the speaker and the receiver is executed.

A process of presenting a determination result regarding the conveyance intention to at least one of the speaker and the receiver is executed.

A program according to an embodiment of the present technology causes a computer system to execute the following steps of:
a step of acquiring character information obtained by converting utterance of a speaker into characters by speech recognition,
a step of determining presence or absence of a conveyance intention that the speaker tries to convey speaker's own utterance content to a receiver using the character information based on a state of the speaker,
a step of executing a process of displaying the character information on display devices used by respective of the speaker and the receiver, and
a step of executing a process of presenting a determination result regarding the conveyance intention to at least one of the speaker and the receiver.

### BRIEF DESCRIPTION OF DRAWINGS

FIG. 1 is a schematic diagram illustrating an outline of a communication system according to an embodiment of the present technology.

FIG. 2 is schematic diagrams each illustrating an exemplary display screen visually recognized by a speaker and a receiver.

FIG. 3 is a block diagram illustrating a configuration example of the communication system.

FIG. 4 is a block diagram illustrating a configuration example of a system control section.

FIG. 5 is a flow chart illustrating an exemplary operation of a speaker side of the communication system.

FIG. 6 is schematic diagrams each illustrating an exemplary process of making a field of view of the speaker difficult to look.

FIG. 7 is a flowchart illustrating an exemplary determination process of a conveyance intention by the character information.

FIG. 8 is a flowchart illustrating the exemplary determination process of the conveyance intention by the character information.

FIG. 9 is a flowchart illustrating the exemplary determination process of the conveyance intention by the character information.

FIG. 10 is a flowchart illustrating the exemplary determination process of the conveyance intention by the character information.

FIG. 11 is a flowchart illustrating the exemplary determination process of the conveyance intention by the character information.

FIG. 12 is a flowchart illustrating the exemplary determination process of the conveyance intention by the character information.

FIG. 13 is schematic diagrams each illustrating an exemplary process of presenting to the speaker that the conveyance intention is absent.

FIG. 14 is a flow chart illustrating an exemplary operation of a receiver side of the communication system.

FIG. 15 is schematic diagrams each illustrating an exemplary process on the receiver side if the conveyance intention is present.

FIG. 16 is schematic diagrams each illustrating an exemplary process on the receiver side if the conveyance intention is absent.

FIG. 17 is schematic diagrams each illustrating a display example of utterance character strings as a comparative example.

FIG. 18 is schematic diagrams each illustrating a display example of utterance character strings as a comparative example.

### MODE(S) FOR CARRYING OUT THE INVENTION

Hereinafter, embodiments according to the present technology will be described with reference to the drawings.

### [Configuration of Communication System]

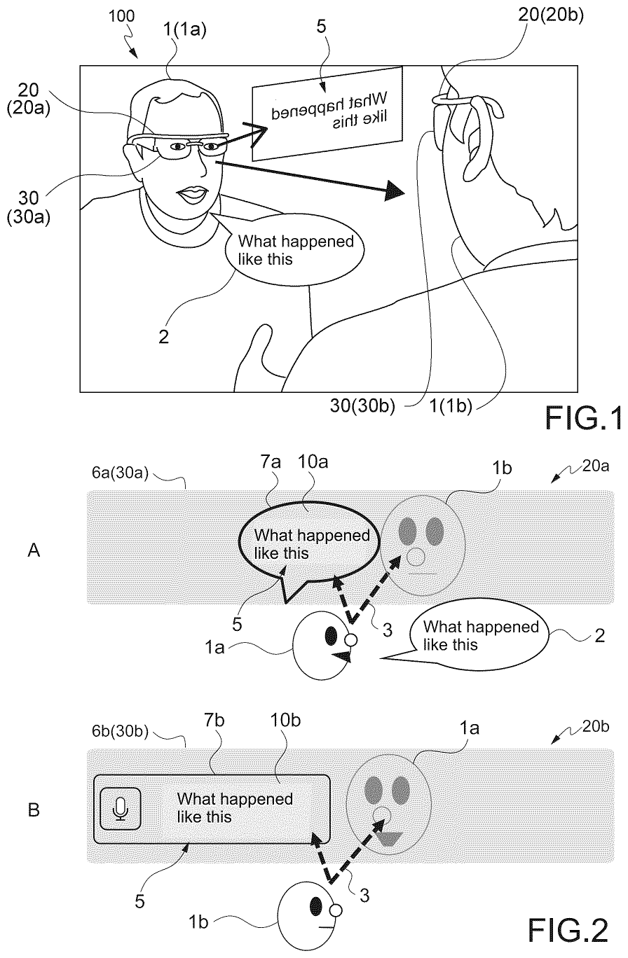

FIG. 1 is a schematic diagram illustrating an outline of a communication system according to an embodiment of the present technology. A communication system 100 is a system that displays character information 5 obtained by speech recognition and supports communication between users 1. The communication system 100 is used, for example, if there are restrictions on hearing.

A situation in which there are restrictions on hearing includes, for example, a case in which a conversation is performed in a noise environment, a case in which a conversation is performed in different languages, a case in which the user 1 has a hearing impairment, or the like. In such a case, it is possible to perform the conversation via the character information 5 by using the communication system 100.

In the communication system 100, a smart glass 20 is used as a device for displaying the character information 5. The smart glass 20 is a spectacle-type HMD (Head Mounted Display) terminal with a transmissive display 30.

The user 1 wearing the smart glass 20 visually recognizes an outside world through the transmissive display 30. At this time, various types of visual information including the character information 5 are displayed on the display 30. As a result, the user 1 can visually recognize the visual information superimposed on a real world, and can confirm the character information 5 during the communication.

In the present embodiment, the smart glass 20 is an example of a transmissive display device.

FIG. 1 schematically illustrates a state of the communication between two users 1a and 1b using the communication system 100. The users 1a and 1b wear smart glasses 20a and 20b, respectively.

In FIG. 1, the speech recognition is executed on a speech 2 of the user 1a, and the character information 5 in which the utterance content of the user 1a is converted into characters is generated. The character information 5 is displayed on both the smart glass 20a used by the user 1a and the smart glass 20b used by the user 1b. As a result, the user 1a and the user 1b communicate with each other via the character information 5.

Hereinafter, it is assumed that the user 1a is a healthy hearing person and the user 1b is a hearing-impaired person. In addition, the user 1a is described as a speaker 1a, and the user 1b is described as a receiver 1b.

FIG. 2 is schematic diagrams each illustrating an exemplary display screen visually recognized by the speaker 1a and the receiver 1b. In “A” of FIG. 2, a display screen 6a displayed on a display 30a of the smart glass 20a worn by the speaker 1a is schematically illustrated. Furthermore, in “B” of FIG. 2, a display screen 6b displayed on a display 30b of the smart glass 20b worn by the receiver 1b is schematically illustrated.

In addition, states in which the lines-of-sights 3 (dotted arrows) of the speaker 1a and the receiver 1b are changed are schematically illustrated in “A” and “B” of FIG. 2. The speaker 1a (receiver 1b) can visually recognize various types of information displayed on the display screen 6a (the display screen 6b) and the state of the outside world looked through the display screen 6a (the display screen 6b) by moving own line-of-sight 3.

In the communication system 100, the speech recognition is performed on the speech 2 made by the speaker 1a, and a character string (the character information 5) indicating the utterance content of the speech 2 is generated. Here, the speaker 1a utters “What happened like this” and a character string “What happened like this” is generated as the character information 5. These pieces of the character information 5 are displayed on the display screens 6a and 6b in real time, respectively.

Note that the displayed character information 5 is the character string obtained by an intermediate result of the speech recognition or a final determination result. Furthermore, the character information 5 does not necessarily match the utterance content of the speaker 1, and an erroneous character string may be displayed.

As shown in “A” of FIG. 2, in the smart glass 20a, the character information 5 obtained by the speech recognition is displayed as it is. In other words, the display screen 6a displays the character string “What happened like this.” In the example shown in “A” of FIG. 2, the character information 5 is displayed inside a balloon-shaped object 7a.

In addition, the speaker 1a can visually recognize the receiver 1b through the display screen 6a. The object 7a including the character information 5 is basically displayed so as not to overlap with the receiver 1b.

As described above, by presenting the character information 5 to the speaker 1a, the speaker 1a can confirm the character information 5 in which speaker's own utterance content is converted into characters. Therefore, if there is an error in the speech recognition, and the character information 5 different from the utterance content of the speaker 1a is displayed or the like, it becomes possible to redo the utterance or to convey to the receiver 1b that the character information 5 is wrong.

In addition, the speaker 1a can confirm the face of the receiver 1b through the display screen 6a (the display 30a), and can realize natural communication.

As shown in “B” of FIG. 2, the character information 5 obtained by the speech recognition is displayed as it is even in the smart glass 20b. In other words, the display screen 6b displays the character string “What happened like this.” In the example illustrating in “B” of FIG. 2, the character information 5 is displayed inside a rectangular object 7b. Furthermore, a microphone icon 8 indicating presence or absence of a speech recognition process or the like is displayed inside the object 7b.

In addition, the receiver 1b can visually recognize the speaker 1a through the display screen 6b. The object 7b including the character information 5 is basically displayed so as not to overlap with the speaker 1a.

As described above, by presenting the character information 5 to the receiver 1b, the receiver 1b can confirm the utterance content of the speaker 1a as the character information 5. Thus, even if the receiver 1b cannot hear the speech 2, it is possible to communicate through the character information 5.

Also, the receiver 1b can confirm the face of the speaker 1a through the display screen 6b (the display 30b). As a result, the receiver 1b can easily confirm information other than the character information such as a motion and an expression of the mouth of the speaker 1a.

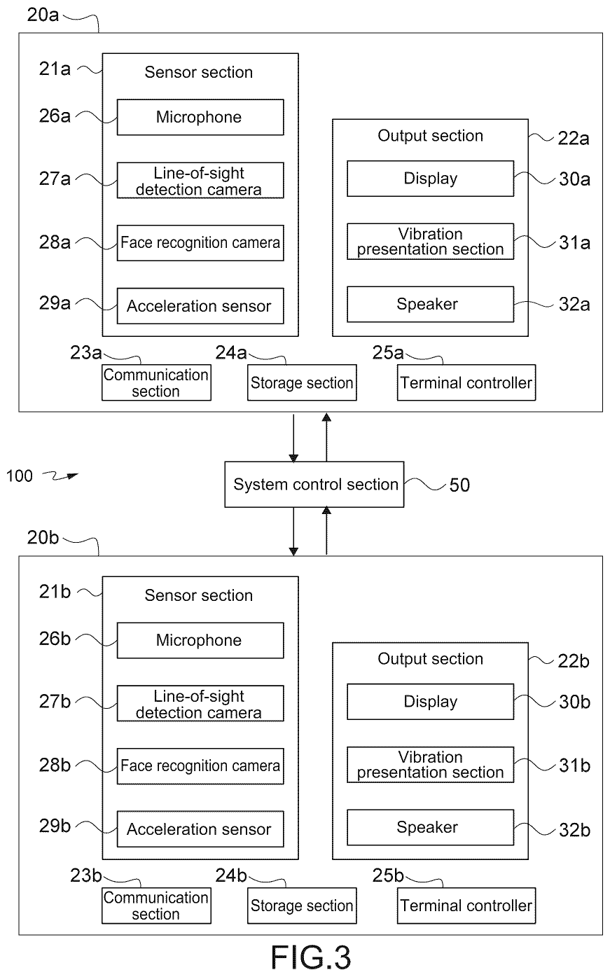

FIG. 3 is a block diagram illustrating a configuration example of the communication system 100. As illustrated in FIG. 3, the communication system 100 includes the smart glass 20a, the smart glass 20b, and a system control section 50.

Here, it is assumed that the smart glass 20a and the smart glass 20b are configured in the same manner, and a configuration of the smart glass 20a is denoted by a symbol “a”, and a configuration of the smart glass 20b is denoted by a symbol “b”.

First, the configuration of the smart glass 20a will be described. The smart glass 20a is a spectacle-type display device, and includes a sensor section 21a, an output section 22a, a communication section 23a, a storage section 24a, and a terminal controller 25a.

The sensor section 21a includes, for example, a plurality of sensor elements provided in a housing of the smart glass 20a, and includes a microphone 26a, a line-of-sight detection camera 27a, a face recognition camera 28a, and an acceleration sensor 29a.

The microphone 26a is a sound collection device that collects the speech 2, and is provided in the housing of the smart glass 20a so as to be able to collect the speech 2 of the wearer (in this case, the speaker 1a).

The line-of-sight detecting camera 27a is an inward-facing camera that captures an image of eyeballs of the wearer. An image of the eyeballs captured by the line-of-sight detection camera 27a is used to detect the line-of-sight 3 of the wearer. The line-of-sight detecting camera 27a is, for example, a digital camera including an image sensor such as a CMOS (Complementary Metal Oxide Semiconductor) and a CCD (Charge Coupled Device). The line-of-sight detection camera 27a may be configured as an infrared camera. In this case, an infrared light source or the like that emits infrared light to the eyeballs of the wearer may be provided. With such a configuration, highly accurate line-of-sight detection can be performed based on the infrared image of the eyeballs.

The face recognition camera 28a is an outward-facing camera that captures an area similar to a field of view of the wearer. The image captured by the face recognition camera 28a is used, for example, to detect a face of a counterpart (here, the receiver 1b) communication with the wearer. The face recognition camera 28a is the digital camera including the image sensor such as the CMOS and the CCD.

The acceleration sensor 29a is a sensor that detects acceleration of the smart glass 20a. The output of the acceleration sensor 29a is used to detect a direction (a posture) of the head of the wearer. As the acceleration sensor 29a, a three-axis acceleration sensor, a three-axis gyro sensor, a nine-axis sensor including a three-axis compass sensor, or the like is used.

The output section 22a includes a plurality of output elements for presenting information and stimuli to the wearer of the smart glass 20a, and includes the display 30a, a vibration presentation section 31a, and a speaker 32a.

The display 30a is a transmissive display device and is fixed to the housing of the smart glass 20a so as to be placed in front of the eyes of the wearer. The display 30a is configured by using a display device such as an LCD (Liquid Crystal Display) and an organic EL display. In the smart glass 20a, for example, a right-eye display and a left-eye display that display images corresponding to respective eyes are provided in a left eye and a right eye of the wearer. Alternatively, a configuration in which a single display is provided to display the same image on both eyes of the wearer, or a configuration in which an image is displayed only on one of the left eye and the right eye of the wearer may be used.

The vibration presentation section 31a is a vibration element that presents vibration to the wearer. As the vibration presentation section 31a, for example, an eccentric motor or an element capable of generating vibration such as a VCM (Voice Coil Motor) is used. The vibration presentation section 31a is provided in the housing of the smart glass 20a, for example. The vibration element provided in other device (such as a portable terminal or a wearable terminal) used by the wearer may be used as the vibration presentation section 31a.

The speaker 32a is a sound reproduction element that reproduces the sound so as to be heard by the wearer. The speaker 32a is configured as a built-in speaker in the housing of the smart glass 20a, for example. The speaker 32a may be configured as an earphone or a headphone used by the wearer.

The communication section 23a is a module used to execute network communication, near-field communication, or the like with another device. As the communication section 23a, for example, a radio LAN module such as a WiFi or a communication module such as Bluetooth (registered trademark) is provided. In addition, a communication module or the like capable of performing communication by a wired connection may be provided.

The storage section 24a is a non-volatile storage device. As the storage section 24a, for example, a recording medium using a solid-state device such as an SSD (Solid State Drive) or a magnetic recording medium such as an HDD (Hard Disk Drive) is used. In addition, the type of the recording medium used as the storage section 24a is not limited, and, for example, any recording medium that non-temporarily records data may be used. The storage section 24a stores programs and the like for controlling operations of the respective sections of the smart glass 20a.

The terminal controller 25a controls the operation of the smart glass 20a. The terminal controller 25a has a hardware configuration required for a computer such as a CPU and a memory (RAM, ROM). Various processes are executed by the CPU such that the programs stored in the storage section 24a are loaded into the RAM and are executed.

Next, a configuration of the 20b is described. The smart glass 20b is a spectacle-type display device, and includes a sensor section 21b, an output section 22b, a communication section 23b, a storage section 24b, and a terminal controller 25b. The sensor section 21b includes a microphone 26b, a line-of-sight detection camera 27b, a face recognition camera 28b, and an acceleration sensor 29b. The output section 22b includes the display 30b, a vibration presentation section 31b, and a speaker 32b.

Each part of the smart glass 20b is configured, for example, in the same manner as each part of the smart glass 20a described above. The description of each part of the smart glass 20a described above can be read as the description of each part of the smart glass 20b by using the wearer as the receiver 1b.

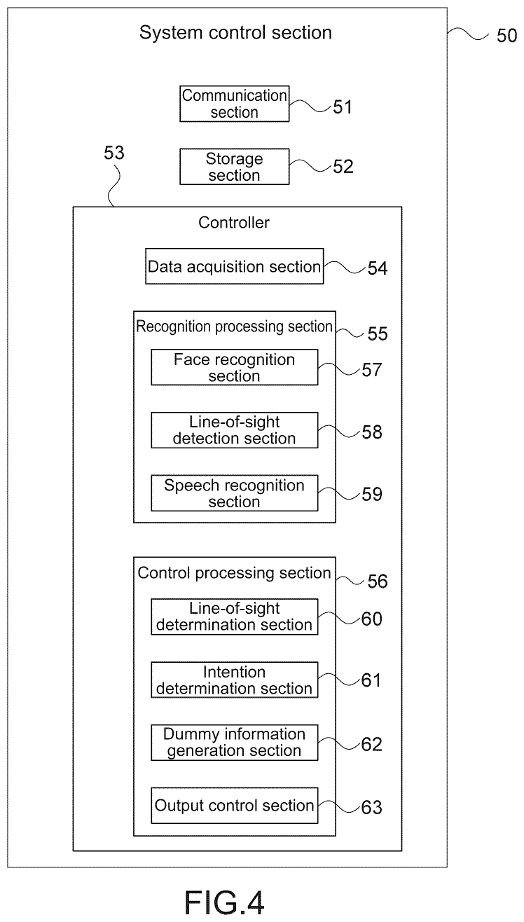

FIG. 4 is a block diagram illustrating a configuration example of the system control section 50. The system control section 50 is a control device that controls overall operations of the communication system 100, and includes a communication section 51, a storage section 52, and a controller 53.

Here, it is assumed that the control section 50 is configured as a server device capable of communicating with the smart glass 20a and the smart glass 20b via a predetermined network. Note that the system control section 50 may be configured by a terminal device (for example, a smartphone or a tablet terminal) that can directly communicate with the smart glass 20a and the smart glass 20b without using the network or the like.

The communication section 51 is a module for executing the network communication, the near field communication, and the like between the system control section 50 and other devices such as the smart glass 20a and the smart glass 20b. As the communication section 51, for example, the radio LAN module such as the WiFi or the communication module such as the Bluetooth (registered trademark) is provided. In addition, the communication module or the like capable of performing communication by the wired connection may be provided.

The storage section 52 is the non-volatile storage device. As the storage section 52, for example, the recording medium using the solid-state element such as the SSD or the magnetic recording medium such as the HDD is used. In addition, the type of the recording medium used as the storage section 52 is not limited, and for example, any recording medium for non-transitory recording of data may be used.

The storage section 52 stores a control program according to the present embodiment. The control program is a program that controls the overall operation of the communication system 100. In addition, the storage section 52 stores a history of the character information 5 obtained by the speech recognition, a log in which the states of the speaker 1a and the receiver 1b during the communication (changes in the line-of-sight 3, the speech speed, the volume, and the like) is recorded, and the like.

In addition, the information stored in the storage section 52 is not limited.

The controller 53 controls the operation of the communication system 100. The controller 53 has a hardware configuration required for the computer such as the CPU and the memory (RAM, ROM). Various processes are executed by the CPU such that the programs stored in the storage section 52 are loaded into the RAM and are executed. The controller 53 corresponds to the information processing apparatus according to the present embodiment.

As the controller 53, for example, devices such as a PLD (Programmable Logic Device) such as an FPGA (Field Programmable Gate Array), or other devices such as an ASIC (Application Specific Integrated Circuit) may be used. Furthermore, a processor such as a GPU (Graphics Processing Unit) may be used.

In the present embodiment, the CPU of the controller 53 executes the program (the control program) according to the present embodiment, so that a data acquisition section 54, a recognition processing section 55, and a control processing section 56 are realized as functional blocks. Then, the information processing method according to the present embodiment is performed by these functional blocks. Note that, in order to implement each functional block, dedicated hardware such as an IC (integrated circuit) may be used, as appropriate.

The data acquisition section 54 appropriately acquires the data necessary for the operations of the recognition processing section 55 and the control processing section 56. For example, audio data, image data, and the like are read from the smart glass 20a or the smart glass 20b via the communication section 51. In addition, data that stores the states of the speaker 1a and the receiver 1b stored in the storage section 52 is read, as appropriate.

The recognition processing section 55 performs various kinds of recognition processes (face recognition, line-of-sight detection, the speech recognition, and the like) based on data outputted from the smart glass 20a or the smart glass 20b.

Among them, the recognition processing section 55 performs the recognition process based on data outputted mainly from the sensor section 21a of the smart glass 20a. Hereinafter, the recognition process based on the sensor section 21a will be mainly described. Note that the recognition process based on the data outputted from the sensor section 21b of the smart glass 20b may be executed as needed.

As illustrated in FIG. 4, the recognition processing section 55 includes a face recognition section 57, a line-of-sight detection section 58, and a speech recognition section 59.

The face recognition section 57 performs a face recognition process on the image data captured by the face recognition camera 28a. That is, the face of the receiver 1b is detected from the image of the field of view of the speaker 1a. In addition, the face recognition section 57 estimates a position and an area of the face of the receiver 1b in the display screen 6a visually recognized by the speaker 1a, for example, based on a face detection result of the receiver 1b (see “A” of FIG. 2). In addition, the face recognition section 57 may estimate a facial expression, a face direction, and the like of the receiver 1b.

A specific method of the face recognition process is not limited. For example, any face detection technique using feature quantity detection, machine learning, or the like may be used.

The line-of-sight detection section 58 detects the line-of-sight 3 of the speaker 1a. Specifically, the line-of-sight 3 of the speaker 1a is detected based on the image data of the eyeballs of the speaker 1a captured by the line-of-sight detection camera 27a. In this process, a vector representing the direction of the line-of-sight 3 may be calculated, or an intersection position (a viewpoint) between the display screen 6a and the line-of-sight 3 may be calculated.

A specific method of the line-of-sight detection process is not limited. For example, if the infrared camera or the like is used as the line-of-sight detection camera 27a, a corneal reflecting method is used. Furthermore, a method of detecting the line-of-sight 3 based on positions of each pupil (iris) may be used.

The speech recognition section 59 performs the speech recognition process based on the audio data obtained by collecting the speech 2 of the speaker 1a. In this process, the utterance content of the speaker 1a is converted into characters and outputted as the character information 5. As described above, the speech recognition section 59 acquires the character information obtained by converting the utterance of the speaker into characters by the speech recognition. In the present embodiment, the speech recognition section 59 corresponds to the acquisition section that acquires the character information.

The audio data used in the speech recognition process is typically data collected by the microphone 26a mounted on the smart glass 20a worn by the speaker 1a. Note that the data collected by the microphone 26b on a receiver 1b side may be used in the speech recognition process of the speaker 1a.

In the present embodiment, the speech recognition section 59 sequentially outputs the character information 5 estimated in the middle of the speech recognition process, in addition to the character information 5 calculated as a final result of the speech recognition process. Therefore, before the character information 5 of the final result is displayed, the character information 5 and the like up to a syllable in the middle thereof are outputted. The character information 5 may be converted into kanji, katakana, alphabet, or the like as appropriate and outputted.

In addition, the reliability of the speech recognition process (the accuracy of the character information 5) may be calculated by the speech recognition section 59 together with the character information 5.

A specific method of the speech recognition process is not limited. Any speech recognition technology such as the speech recognition using an acoustic model or a language model and the speech recognition using the machine learning may be used.

The control processing section 56 performs various processes for controlling the operation of the smart glass 20a or the smart glass 20b.

As illustrated in FIG. 4, the control processing section 56 includes a line-of-sight determination section 60, an intention determination section 61, a dummy information generation section 62, and an output control section 63.

The line-of-sight determination section 60 executes a determination process regarding the line-of-sight 3 of the speaker 1a based on the detection result of the line-of-sight detection section 58.

Specifically, the line-of-sight determination section 60 determines whether or not the line-of-sight 3 of the speaker 1a is deviated from an area in which the character information 5 is displayed in the smart glass 20a used by the speaker 1a based on the detected line-of-sight 3 of the speaker 1a.

Hereinafter, the area in which the character information 5 is displayed in the smart glass 20a (the display screen 6a) will be referred to as a character display area 10a on a speaker 1a side. The character display area 10a is an area including the character string that is the character information 5 inside, and is appropriately set as an area on the display screen 6a. For example, an area inside the balloon-shaped object 7a described with reference to “A” of FIG. 2 is set as the character display area 10a.

A position, a size, and a shape of the character display area 10a may be fixed or may be variable. For example, the character area 10a may be resized or reshaped depending on a length and the number of columns of the character string. Furthermore, for example, the position of the character display area 10a may be changed so as not to overlap with the position of the face of the receiver 1b in the display screen 6a.

In addition, the area in which the character information 5 is displayed in the smart glass 20b (the display screen 6b) is referred to as a character display area 10b on the receiver 1b side. For example, an area inside the rectangular object 7b described with reference to “B” of FIG. 2 is set as the character display area 10b.

The line-of-sight determination section 60 reads information (the position, the shape, the size, and the like) of the character display area 10a, and determines whether or not the line-of-sight 3 of the speaker 1a is directed to the character display area 10a. Thus, it is possible to identify whether or not the speaker 1a looks at the character information 5.

The determination result by the line-of-sight determination section 60 is outputted to the intention determination section 61 and the output control section 63, as appropriate.

Based on the state of the speaker 1a, the intention determination section 61 determines presence or absence of a conveyance intention that the speaker 1a tries to convey speaker's own utterance content to the receiver 1b using the character information 5. In the present embodiment, the intention determination section 61 corresponds to the determination section that determines the presence or absence of the conveyance intention.

Here, the conveyance intention is an intention of the speaker 1a to convey the utterance content to the receiver 1b using the character information 5. This can be said to be intended to properly convey the utterance content to, for example, the receiver 1b who cannot hear the speech 2. It can also be said that the determination of the presence or absence of the conveyance intention is to determine whether or not the speaker 1a performs conscious communication using the character information 5.

In the intention determination section 61, whether or not the speaker 1a is communicating with such a conveyance intention is determined by referring to the state of the speaker 1a.

In the present embodiment, if the line-of-sight 3 of the speaker 1a deviates from the area (the character display area 10a) in which the character information 5 is displayed, the intention determination section 61 starts the determination process of the conveyance intention. That is, if the line-of-sight determination section 60 determines that the line-of-sight 3 of the speaker 1a is not directed to the character display area 10a, the determination process by the intention determination section 61 is started.

For example, if the speaker 1a takes eyes off from the character display area 10a, the speaker 1a cannot confirm the correctness or the like of the character information 5. In such a situation, there is a possibility that the conveyance intention using the character information 5 is lost in the speaker 1a. Conversely, if the speaker 1a looks at the character display area 10a, since the speaker 1a focuses on the character information 5, it can be estimated that it has the conveyance intention using the character information 5.

Note that even if the speaker 1a takes eyes off from the character display area 10a, it does not necessarily mean that the conveyance intention is absent using the character information 5 for the speaker 1a. For example, the speaker 1a may simply confirm the face of the receiver 1.

Therefore, the intention determination section 61 determines the conveyance intention by triggering that the line-of-sight 3 of the speaker 1a deviates from the character display area 10a. This eliminates the need to perform unnecessary determination process. In addition, it is possible to immediately detect a state that the conveyance intention is absent for the speaker 1a.

The dummy information generation section 62 generates dummy information that makes the speaker 1a appear to be uttering even if there is no speech 2 of the speaker 1a.

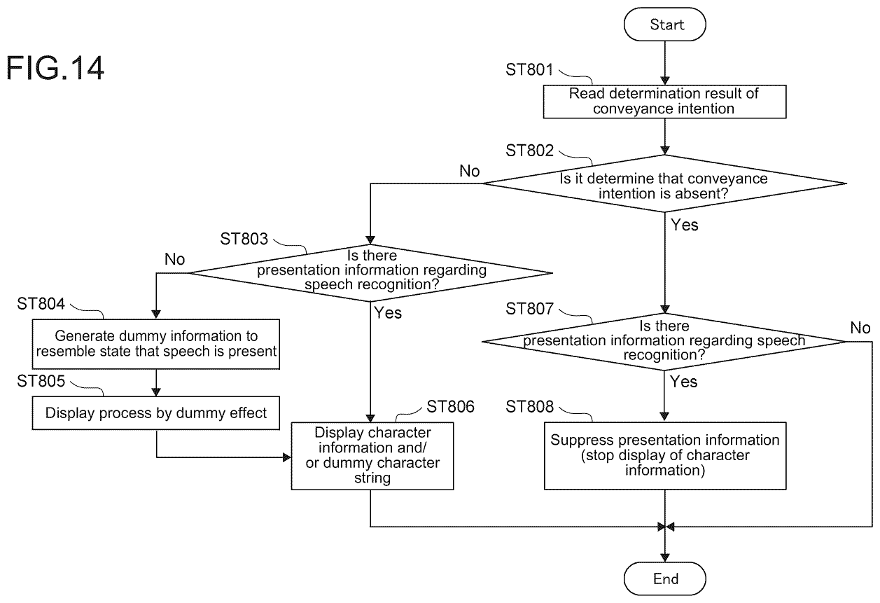

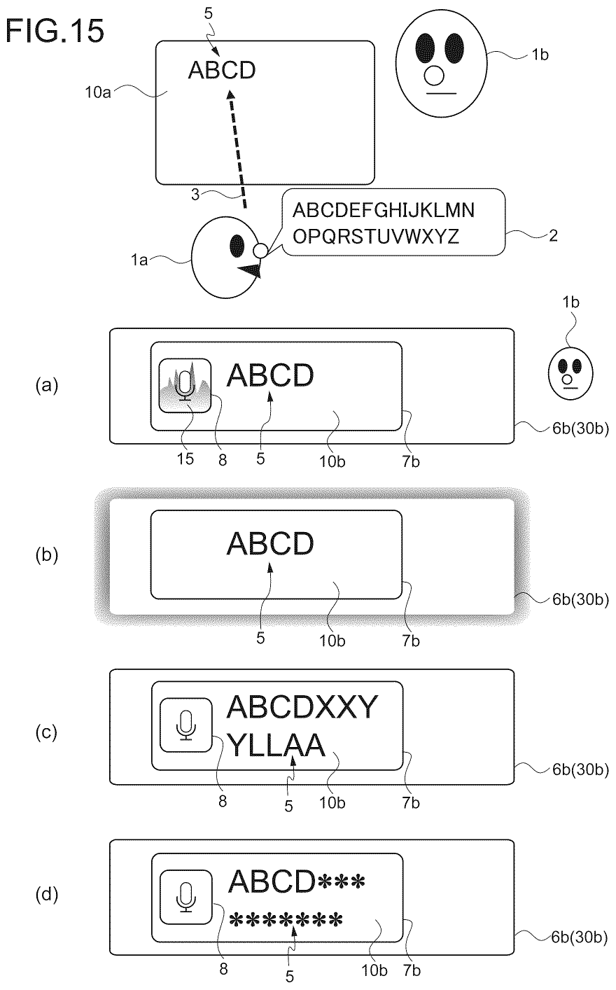

The dummy information which is dummy character information is generated. The dummy information is, for example, information such as a character string displayed in place of the original character information 5 on the screen of the receiver 1b and a direction (an effect) that the speaker 1a appears to be uttering. The dummy information generated is outputted to the smart glass 20b. A display control using the dummy information and the like will be described in detail later with reference to FIGS. 14 and 15.

The output control section 63 controls the operations of the output section 22a provided in the smart glass 20a and the output section 22b provided in the smart glass 20b.

Specifically, the output control section 63 generates data to be displayed on the display 30a (the display 30b). The generated data is outputted to the smart glass 20a (the smart glass 20b), and the display on the display 30a (the display 30b) is controlled. The data includes data of the character information 5, data for designating a display position of the character information 5, and the like. That is, it can be said that the output control section 63 performs the display control on the display 30a (the display 30b). As described above, the output control section 63 executes a process of displaying the character information 5 on the smart glass 20a and the smart glass 20b used by each of the speaker 1a and the receiver 1b.

Furthermore, the output control section 63 generates, for example, vibration data specifying a vibration pattern of the vibration presentation section 31a (the vibration presentation section 31b), sound data reproduced by the speaker 32a (the speaker 32b), and the like. By using the vibration data and the sound data, presentation of vibration and reproduction of sound in the smart glass 20a (the smart glass 20b) are controlled.

Furthermore, the output control section 63 executes a process of presenting the determination result regarding the conveyance intention to the speaker 1a and the receiver 1b. Specifically, the output control section 63 acquires the determination result of the conveyance intention by the intention determination section 61 described above. Then, the output section 22a (the output section 22b) mounted on the smart glass 20a (the smart glass 20b) is controlled to present the determination result of the conveyance intention to the speaker 1a (the receiver 1b).

In the present embodiment, if it determines that the conveyance intention is absent, the output control section 63 generates notification data notifying that the conveyance intention is absent to the speaker 1a and the receiver 1b. The notification data is outputted to the smart glass 20a (the smart glass 20b), and the output section 22a (the output section 22b) is driven in accordance with the notification data. As a result, the speaker 1a can be made aware of, for example, a situation in which the conveyance intention by the character information has disappeared (decreased). In addition, the receiver 1b can be notified that, for example, the speaker 1a is uttering without conveyance intention by the character information.

The notification data includes at least one of visual data, haptic data, and sound data.

The visual data is data for visually conveying that the conveyance intention is absent. As the visual data, for example, data of an image (an icon or the display screen 6a) displayed on the display 30a (the display 30b) and indicating that the conveyance intention is absent is generated. Alternatively, data specifying an icon, a visual effect, or the like indicating that the conveyance intention is absent may be generated.

The haptic data is data for conveying that the conveyance intention is absent by haptic such as vibration. In the present embodiment, data for vibrating the vibration presentation section 31a (the vibration presentation section 31b) is generated.

The sound data is data for conveying, by a warning sound or the like, that the conveyance intention is absent. In the present embodiment, data reproduced by the speaker 32a (the speaker 32b) is generated.

The type, the number, and the like of the notification data are not limited, and, for example, two or more types of notification data may be used in combination. A method of presenting that the conveyance intention is absent will be described in detail later.

As described above, the system control section 50 is configured as the server device or the terminal device. However, the configuration of the system control section 50 is not limited thereto.

For example, the smart glass 20a (the smart glass 20b) may constitute the control section 50. In this case, the communication section 23a (the communication section 23b) functions as the communication section 51, the storage section 24a (the storage section 24b) functions as the storage section 52, and the terminal controller 25a (the terminal controller 25b) functions as the controller 53. In addition, the functions of the system control section 50 (the controller 53) may be distributed. For example, the speech recognition section 59 may be realized by a server device dedicated to speech recognition.

### [Operation of Communication System on Speaker Side]

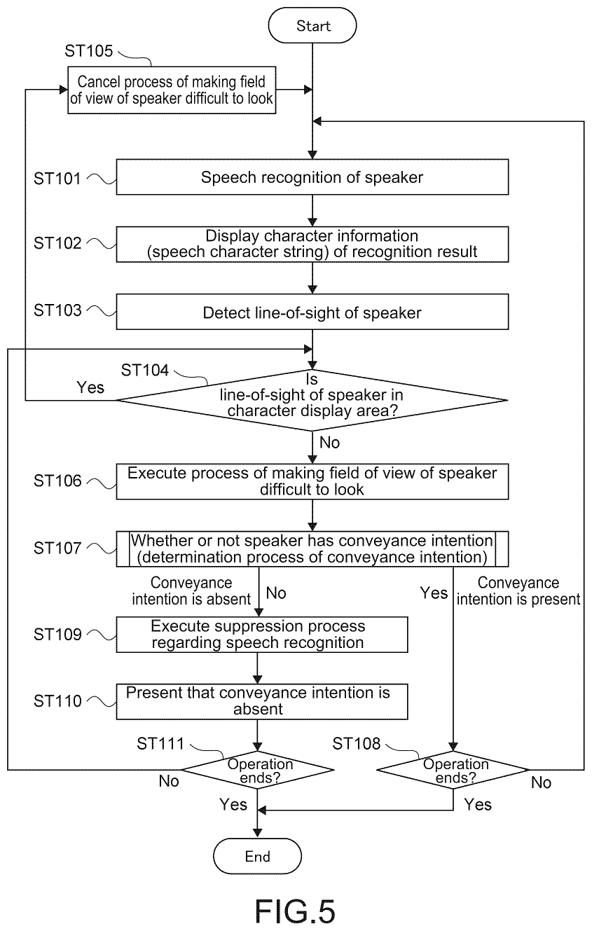

FIG. 5 is a flow chart illustrating an exemplary operation of the speaker 1a side of the communication system 100. The process illustrated in FIG. 5 is a process for controlling the operation of the smart glass 1a mainly used by the speaker 20a, and is repeatedly executed while the speaker 1a and the receiver 1b communication with each other. The operation of the communication system 100 with respect to the speaker 1a will be described below with reference to FIG. 5.

First, the speech recognition is performed on the speech 2 of the speaker 1 (Step 101). For example, the speech 2 made by the speaker 1a is collected by the microphone 26a of the smart glass 20a. The collected data is inputted to the speech recognition section 59 of the system control section 50. In the speech recognition section 59, the speech recognition process is executed on the speech 2 of the speaker 1a, and the character information 5 is outputted. The character information 5 is a text of a recognition result about the speech 2 of the speaker 1a, and is a utterance character string in which the utterance content is estimated.

Next, the character information 5 (the utterance character string) which is the recognition result of the speech recognition is displayed (Step 102). The character information 5 outputted from the speech recognition section 59 is outputted to the smart glass 20a via the output control section 63, and is displayed on the display 30a visually recognized by the speaker 1a. Similarly, the character information 5 is output to the smart glass 20b via the output control section 63, and is displayed on the display 30b visually recognized by the receiver 1b.

Note that the character information 5 displayed here may be the character string as a result in the middle of the speech recognition, or may be an erroneous character string misrecognized in the speech recognition.

Next, the line-of-sight 3 of the speaker 1a is detected (Step 103). Specifically, a vector indicating the line-of-sight 3 of the speaker 1a is estimated by the line-of-sight detection section 58 based on the images of the eyeballs of the speaker 1a captured by the line-of-sight detection camera 27a. Alternatively, the position of the viewpoint in the display screen 6a may be estimated. The detected line-of-sight 3 of the speaker 1a is outputted to the line-of-sight determination section 60.

Next, the line-of-sight determination section 60 determines whether or not the line-of-sight 3 (the viewpoint) of the speaker 1a is in the character display area 10a (Step 103). For example, if the vector indicating the line-of-sight 3 of the speaker 1a is estimated, it determines whether or not the estimated vector intersects the character display area 10a. Furthermore, for example, if the viewpoint of the speaker 1a is estimated, it determines whether or not the position of the viewpoint is included in the character display area 10a.

If it determines that the line-of-sight 3 of the speaker 1a is in the character display area 10a (Yes in Step 104), the processes of Step 101 and subsequent steps are executed again assuming that the speaker 1a looks at the character information 5. If the process executed in Step 106 described below continues, the process is canceled (Step 105).

If it determines that the line-of-sight 3 of the speaker 1a is not in the character display area 10a (No in Step 104), the output control section 63 executes a process of making the field of view of the speaker 1a difficult to look (Step 106).

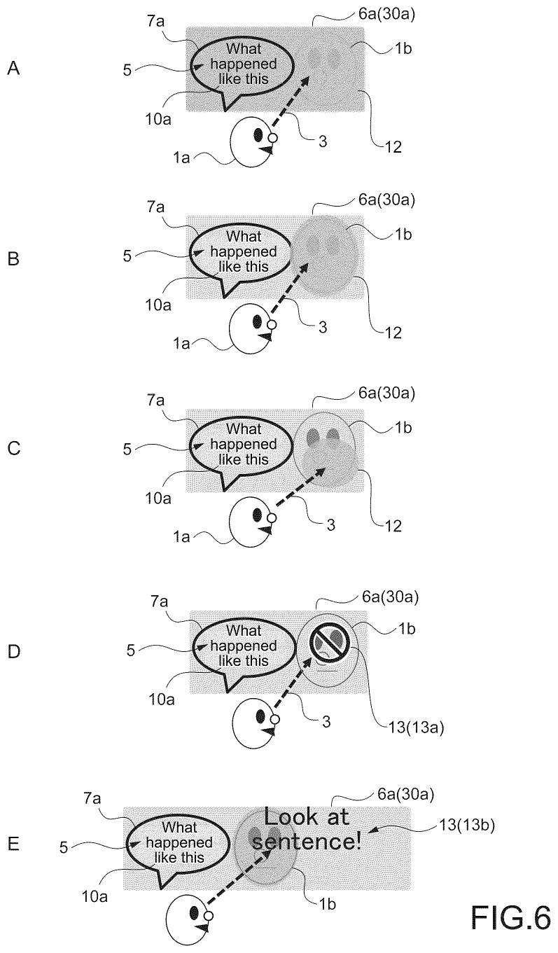

The state that the line-of-sight 3 of the speaker 1a is not in the character display area 10a is, for example, a state that the speaker 1a looks at the face of the receiver 1b or a state other than the utterance character string such as an own hand. In such cases, the output control section 63 controls the display 30a to make a presentation state that an entire screen looked by the speaker 1a and a periphery of a viewpoint position are difficult to look (see FIG. 6).

As described above, if the line-of-sight 3 of the speaker 1a deviates from the character display area 10a in which the character information 5 is displayed, the output control section 63 executes the process of making the field of view of the speaker 1a difficult to look. This process makes it difficult for the speaker 1a to visually recognize the face of the counterpart or surrounding objects. By creating such a state, it becomes possible to give a sense of discomfort to the speaker 1a who takes eyes off from the character information 5.

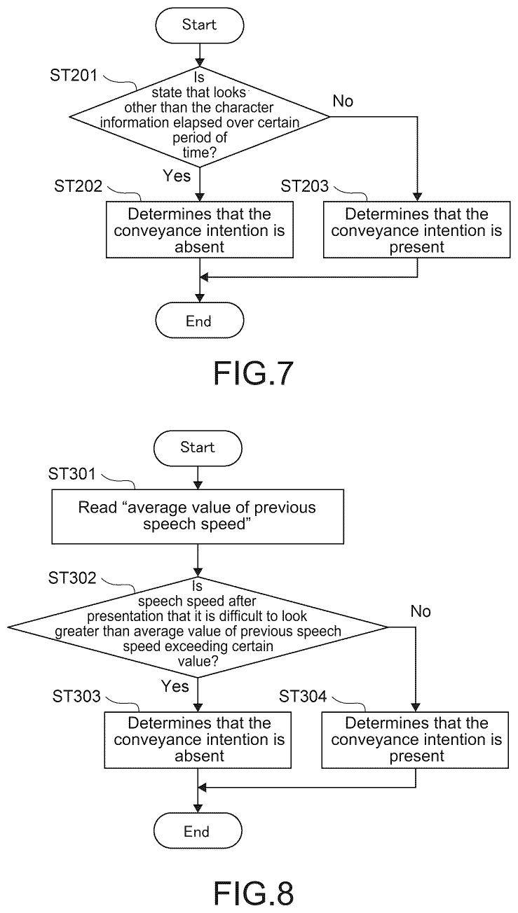

If the process of making the field of view of the speaker 1a difficult to look is executed, the intention determination section 61 determines whether or not the speaker 1a has the conveyance intention using the character information 5 (Step 107). In the intention determination section 61, various parameters (the line-of-sight 3, the speech speed, the volume, and the like at the time of utterance) indicating the state of the speaker 1a are appropriately read. Then, it determines whether or not the read parameters satisfy a determination condition indicating that the speaker 1a has no conveyance intention (see FIGS. 7 to 12).

In this case, it determines that the conveyance intention is present until the determination condition is satisfied. In addition, once the determination condition is satisfied, it determines that the conveyance intention is absent.

If it determines that the speaker 1a has the conveyance intention using the character information 5 (Yes in Step 107), it determines whether or not the operation of the communication system 100 is terminated (Step 108).

For example, if the communication between the speaker 1a and the receiver 1b is ended and the operation of the system is stopped, it determines that the operation is ended (Yes in Step 108), and the entire processes are ended.

In addition, for example, if the speaker 1a and the receiver 1b continue to communicate with each other and the operation of the system continues, it determines that the operation is not ended (No in Step 108), and the processes after Step 101 and subsequent steps are executed again.

At the time when the determination process of the conveyance intention is executed, the process of making the field of view of the speaker 1a difficult to look continues. Therefore, unless the speaker 1a returns the line-of-sight to the character information 5 (the character display area 10a), the process of making the field of view difficult to look is not canceled even if it determines that the conveyance intention is present. From another point of view, when the line-of-sight 3 of the speaker 1a begins to read the utterance character string again (Yes in Step 104), Step 105 is executed to reset the presentation state that it is difficult to look.

As described above, in the present embodiment, if the line-of-sight 3 of the speaker 1a is returned to the character display area 10a in which the character information 5 is displayed, the process of making the field of view of the speaker 1a difficult to look is canceled.

As described above, if the speaker 1a deviates the line-of-sight 3 from the character information 5, the speaker 1a is given a sense of discomfort that the field of view becomes difficult to look. If the speaker 1a returns the line-of-sight 3 to the character information 5, the process of making it difficult to look is canceled, so that it is possible to naturally guide the speaker 1a to look at the character information 5.

Returning to Step 107, if it determines that the speaker 1a has no conveyance intention using the character information 5 (No in Step 107), the output control section 63 executes a suppression process regarding the speech recognition (Step 108). In the present disclosure, in the suppression process regarding the speech recognition, controls such as stopping the process and reducing the frequency of the process are performed with respect to the process regarding the speech recognition.

In the present embodiment, the speech recognition process is stopped as the suppression process. As a result, the character information 5 is not newly updated during the period in which it determines that the conveyance intention is absent.

Furthermore, as the suppression process, the process of displaying the character information 5 may be stopped in at least one of the smart glass 20a and 20b used by each of the speaker 1a and the receiver 1b. In this case, the process itself of the speech recognition continues in the background.

For example, in the state that the speaker 1a has no conveyance intention, even if the result of the speech recognition (the character information 5) is incorrect, the incorrect result is directly conveyed to the receiver 1b. As a result, there is a possibility that the receiver 1b is confused by displaying the character information 5. In order to avoid such a situation, in the present embodiment, if the conveyance intention is absent, updating and displaying of the character information 5 are stopped. Accordingly, a burden on the receiver 1b can be sufficiently reduced.

For example, if the speech recognition process itself is stopped as described above, a process load and a communication load can be reduced. When only the display of the character information 5 is stopped, the speech recognition continues. For this reason, if the speaker 1a resumes the communication with consciousness about the character information 5 (with the conveyance intention), the character information 5 can be immediately resumed to be displayed.

Once the suppression process of the speech recognition is executed, the output control section 63 presents to the speaker 1a oneself that the conveyance intention is absent (Step 110). In the present embodiment, notification data is generated to notify the speaker 1a that the conveyance intention is absent, and is outputted to the smart glass 20a. Then, it is presented that the conveyance intention is absent via the display 30a, the vibration presentation section 31a, the speaker 32a, and the like of the smart glass 20a.

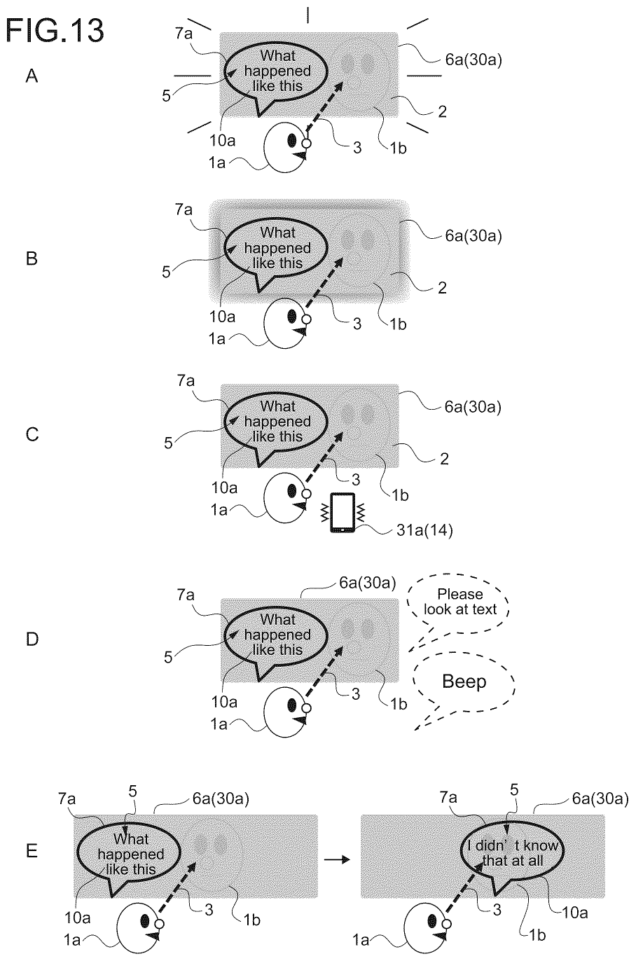

A method of presenting that the conveyance intention is absent will be described later with reference to FIG. 13.

Once it is presented to the speaker 1a that the conveyance intention is absent, it determines whether or not the operation of the communication system 100 is ended (Step 111). This determination process is the same as the determination process of Step 108.

For example, if it determines that the operation ends (Yes in Step 111), the entire process ends. Furthermore, for example, if it determines that the operation is not ended (No in Step 111), the processes of Step 104 and subsequent steps are executed again.

As described above, if it determines that the conveyance intention is absent, the suppression process regarding the speech recognition (Step 109) and the process of presenting that the conveyance intention is absent (Step 110) are respectively executed until the speaker 1a returns the line-of-sight to the character display area 10a or it determines that the conveyance intention is present. If it determines in Step 104 that the speaker 1a has returned the line-of-sight to the character display area 10a and if it determines in Step 107 that the conveyance intention is present, the processes of Steps 109 and 110 are canceled, and the normal speech recognition and display control are resumed.

### [Process of Making Field of View of Speaker Difficult to Look]

FIG. 6 is schematic diagrams each illustrating an exemplary process of making the field of view of the speaker 1a difficult to look. “A” to “E” of FIG. 6 each schematically illustrates an exemplary display screen 6a displayed on the display 30a by the process of making the field of view of the speaker 1a difficult to look, which is executed in Step 106 of FIG. 5. Hereinafter, with reference to “A” to “E” of FIG. 6, the process of making the field of view of the speaker 1a difficult to look will be described in detail.

In the present embodiment, as the process of making the field of view of the speaker 1a difficult to look, a process of decreasing the transparency of at least a part of a transmissive display 30a (the display screen 6a) is executed. As the transparency of the display 30a decreases, it becomes difficult for the speaker 1a to visually recognize scenery of the outside world or the receiver 1b that has been looked through the display 30a.

“A” of FIG. 6 illustrates that the transparency of the entire screen of the display screen 6a is decreased. In this case, for example, a shield image 12 for decreasing the transparency is displayed on the entire display screen 6a. As a result, the entire field of view of the speaker 1a becomes difficult to look.

In “A” of FIG. 6, since the displaying of the object 7a on which the character information 5 is displayed is not changed, the speaker 1a can easily visually recognize the character information 5, and the line-of-sight 3 of the speaker 1a is easily guided to the character information 5.

Furthermore, for example, the object 7a and the shield image 12 have a similar color, to thereby make the object 7a (the character information 5) difficult to look. As a result, it is possible to sufficiently warn the speaker 1a that the line-of-sight 3 is deviated from the character information 5 (the character display area 10a).

“B” of FIG. 6 is an example of decreasing the transparency of a face area of the receiver 1b in the display screen 6a (an area in which the face of the receiver 1b is looked through the display 30a). In this case, for example, the shield image 12 for decreasing the transparency is displayed in the face area of the receiver 1b estimated by the face recognition section 57. As a result, it is difficult to look at the face of the receiver 1b looked from the speaker 1a. Thus, for example, it becomes possible to effectively give a sense of discomfort to the speaker 1a if the speaker 1a continues to utter while paying attention to the face of the receiver 1b.

“C” of FIG. 6 is an example of decreasing the transparency with respect to the position of the line-of-sight 3 (the viewpoint) of the speaker 1a in the display screen 6a. In this case, for example, the shield image 12 having a predetermined size is displayed around the viewpoint of the speaker 1a estimated by the line-of-sight detection section 58. This makes it difficult for the speaker 1a to look at a target of interest. Thus, for example, it becomes possible to effectively give a sense of discomfort to the speaker 1a if the speaker 1a focuses on any target other than the character information 5 (for example, the speaker 1a's own hand, the face or the background of the receiver 1b, and the like).

In the present embodiment, the process of gradually decreasing the transparency of the display 30a is executed. For example, while the process of making the field of view of the speaker 1a difficult to look is being executed, the process of gradually decreasing the transparency of the shield image 12 (the process of gradually darkening a color of the shielding image 12) is executed.

As a result, the longer the line-of-sight 3 of the speaker 1a is deviated from the character information 5 (the character display area 10a), the more difficult the field of view becomes to look. On the other hand, if a period in which the line-of-sight 3 of the speaker 1a is deviated is short, a change in the field of view is small. By controlling the transparency in this way, it is possible to warn the speaker 1a that the character information 5 is not looked without unnecessarily giving a sense of discomfort.

A method of decreasing the transparency of the display 30a is not limited to the above-described method of using the shield image 12. For example, if a dimming device or the like for adjusting an amount of transmitted light is provided in the display 30a, the transparency may be adjusted by controlling the dimming device.

As the process of making the field of view of the speaker 1a difficult to look, a process of displaying an object that blocks the field of view of the speaker 1a on the transmissive display 30a may be executed. Hereinafter, the object that blocks the field of view of the speaker 1a will be referred to as a shield object 13. By displaying the shield object 13, the speaker 1a becomes difficult to visually recognize scenery of the outside world or the receiver 1b that has been looked through the display 30a.

“D” of FIG. 6 is an example that a warning icon 13a is displayed as the shield object 13 on the display screen 6a. The warning icon 13a is a UI icon for warning that the speaker 1a is paying attention to other than the character information 5. A design and the like of the warning icon 13a are not limited.

In “D” of FIG. 6, the warning icon 13a is displayed depending on the position and the area of the face of the receiver 1b. For example, a display position and a display size of the warning icon 13a are set so as to cover the face of the receiver 1b. As a result, the face of the receiver 1b becomes difficult to look, and it becomes possible to sufficiently give a sense of discomfort to the speaker 1a.

In addition, the warning icon 13a may be displayed depending on the viewpoint of the speaker 1a.

The alert icon 13a may be displayed as an animated icon or may be displayed so as to move in the display screen 6a.

In “E” of FIG. 6, a warning character string 13b is displayed as the shield object 13 on the display screen 6a. The warning character string 13b is a character string for warning in a sentence that the speaker 1a focuses on other than the character information 5. The content, design, and the like of the warning string 13b are not limited.

In “E” of FIG. 6, the warning character string 13b is displayed depending on the position and the area of the face of the receiver 1b. For example, a display position and a display size of the warning character string 13b are set so as to cover the face of the receiver 1b. As a result, the face of the receiver 1b becomes difficult to look, and it becomes possible to sufficiently give a sense of discomfort to the speaker 1a.

In addition, the warning character string 13b may be displayed depending on the viewpoint of the speaker 1a.

In addition, the warning character string 13b may be displayed as an animated character string, or may be displayed so as to move in the display screen 6a.

Furthermore, in the present embodiment, a process of gradually displaying the shield object 13 (the warning icon 13a or the warning character string 13b) is executed. For example, while the process of making the field of view of the speaker 1a difficult to look is being executed, the process of gradually decreasing the transparency of the shield object 13 (the process of gradually darkening a color of the shield object 13) is executed.

As a result, the longer the line-of-sight 3 of the speaker 1a is deviated from the character information 5 (the character display area 10a), the better the shield object 13 can be looked, and the more difficult the field of view of the speaker 1a becomes to look. On the other hand, if the time period in which the line-of-sight 3 of the speaker 1a is deviated is short, since the shield object 13 is not conspicuous, the change in the field of view is small. By controlling the display of the shield object 13 in this way, it is possible to warn the speaker 1a that the character information 5 is not looked without unnecessarily giving a sense of discomfort.

In the present embodiment, the process of making the field of view of the speaker 1a difficult to look is appropriately adjusted.

Hereinafter, a process of setting a speed at which the field of view is difficult to look will be mainly described. It should be noted that it is also possible to adjust a degree of difficult to look, the content of the process making difficult to look, and the like.

The speed at which the field of view becomes difficult to look is, for example, a speed at which the field of view increases, and is a speed at which the transparency of the shield image 12 and the shield object 13 decreases.

For example, if the speaker 1a is warned quickly that the character information 5 is not looked, the speed at which the field of view becomes difficult to look is set high. On the other hand, if it is not necessary to hurry the warning, the speed at which the field of view is difficult to look is set low.

For example, based on reliability (Confidence Level) of the speech recognition, the speed that makes the field of view of the speaker 1 difficult to look is set. The reliability of the speech recognition is, for example, an index indicating correctness of the character information 5. The higher the reliability is, the more likely the character information 5 represents the correct utterance content. Note that the reliability of the speech recognition is outputted from the speech recognition section 59 together with the character information 5.

In the present embodiment, inversely to the reliability of the speech recognition, the process of making the field of view of the speaker 1a difficult to look is executed.

For example, when the reliability is low, the speed of decreasing the transparency is increased depending on the reliability, so that the field of view of the speaker 1a becomes opaque at once. Thus, the speaker 1a can be immediately checked if the wrong character information 5 is displayed or the like.

Furthermore, for example, when the reliability of the speech recognition is high, the speed of decreasing the transparency is decreased, so that it becomes slowly opaque. Thus, if the correct character information 5 is displayed or the like, a sense of discomfort will not be unnecessarily given to the speaker 1a.

Furthermore, a speed at which the field of view of the speaker 1a is difficult to look may be set based on the speech speed of the speaker 1a. The speech speed of the speaker 1a is calculated, for example, by the speech recognition section 59 based on the characters (words) or the like made per unit time.

In the present embodiment, a process of learning a way of speaking by the speaker 1a on a personal basis and making the field of view difficult to look depending on the way of speaking by the speaker 1a is executed. The way of speaking by the speaker 1a is stored in the storage section 52 for each speaker 1a.

For example, for the speaker 1a that has been learned to speak quickly, the transparency is made much lower (the speed of decreasing the transparency is increased) so that the field of view becomes difficult to look quickly. Thus, for example, a situation in which a large amount of wrong character information 5 is presented to the receiver 1b can be avoided.

Furthermore, for example, in the case of the speaker 1a that is slow in speech speed, it is not necessary to urge confirmation of the character information 5 as compared with the case of a speaker that is fast in speech speed, so that the speed of decreasing the transparency is reduced. Thus, a sense of discomfort will not be unnecessarily given to the speaker 1a.

Furthermore, the speed at which the field of view of the speaker 1a is difficult to be looked may be set based on a motion tendency of the line-of-sight 3 of the speaker 1a. The motion tendency of the line-of-sight 3 of the speaker 1a is estimated based on, for example, a history of the line-of-sight 3 detected by the line-of-sight detection section 58.

In the present embodiment, with respect to the line-of-sight 3 of the speaker 1a, a process is executed in which a degree of return from the position of the face or the like of the receiver 1b to the position of the character information 5 (the utterance character string) is learned on the personal basis, and the field of view is made difficult to look depending on the degree of return of the line-of-sight 3 to the character information 5. It should be noted that data of the degree of return of the line-of-sight 3 to the character information 5 is stored in the storage section 52 for each speaker 1a.

For example, with respect to the speaker 1a in which the line-of-sight returns quickly to the character information 5, it is considered that the line-of-sight 3 moves so as to immediately confirm the character information 5 even if there is no warning or the like, so that it becomes slowly opaque (decreasing the speed of decreasing the transparency). Thus, a sense of discomfort will not be unnecessarily given to the speaker 1a.

Furthermore, for example, with respect to the speaker 1a in which the line-of-sight returns slowly to the character information 5, it is desired to make the speaker 1a notice quickly that the line-of-sight 3 is deviated from the character information 5, so that it becomes opaque quickly (increasing the speed of decreasing the transparency). This makes it possible to immediately confirm the character information 5.

In addition, based on a noise level around the speaker 1a, the speed at which the field of view of the speaker 1a is difficult to look may be set. The noise level is, for example, acoustic information such as a volume and a sound pressure of the noise, and is estimated by the speech recognition section 59 based on the data collected by the microphone 26a (or the microphone 26b).

In the present embodiment, the process of making the field of view difficult to look is executed depending on the acoustic information (the noise level) of the surrounding noise.

For example, in a place where the noise level is high, there is a possibility that the reliability of speech recognition or the like is lowered and a misrecognition result may be displayed as the character information 5. For this reason, it is desired to make the speaker 1a notice quickly that the line-of-sight 3 is deviated from the character information 5, so that it becomes opaque quickly. This makes it possible to immediately confirm the character information 5. On the other hand, in a place where the noise level is low, it is not necessary to urge confirmation of the character information 5 as compared with the case in which the noise level is high, and therefore, the speed of decreasing the transparency is set to be small.

Furthermore, in the process of making the field of view of the speaker 1a difficult to look, the degree of difficult to look may be changed stepwise. For example, if a state that the line-of-sight 3 of the speaker 1a is deviated from the character information 5 (the character display area 10a) continues, a type of the process of making the field of view difficult to look is changed. Typically, the longer the time when the line-of-sight 3 is away from the character information 5, the more the process having a higher degree of difficult to look is executed.

For example, a process of decreasing the transparency (see “A”, “B”, and “C” of FIG. 6) is executed at first, and when the line-of-sight 3 of the speaker 1a is still unchanged and looks other than the character information 5, the shield object 13 is displayed and not looked (see “D” and “E” of FIG. 6). As described above, by dividing the display for making it difficult to look into a plurality of steps, it is possible to reliably convey the speaker 1a that the line-of-sight 3 is deviated from the character information 5.

### [Determination Process of Conveyance Intention]

FIG. 7 to FIG. 12 are flowcharts each illustrating exemplary process of determining the conveyance intention by the character information 5. These processes are internal processes in Step 107 of FIG. 5, and are processes for determining whether or not each of the processes satisfies the determination condition indicating that the speaker 1a has no conveyance intention.

In the present embodiment, the determination process each illustrated in FIGS. 7 to 12 is executed in parallel. That is, if at least one of the determination conditions illustrated in FIGS. 7 to 12 is satisfied, it determines that the speaker 1a has no conveyance intention by the character information 5.

Hereinafter, the determination process of the conveyance intention will be described in detail with reference to FIGS. 7 to 12.

In FIG. 7, the determination process of the conveyance intention is executed based on the line-of-sight 3 of the speaker 1a. In this determination process, a condition (hereinafter, referred to as a determination condition C1) is determined based on the line-of-sight 3 of the speaker 1a in which the speaker 10a continuously looks other than the character information 5 (the character display area 10a) and a certain period of time elapses.

First, it determines whether or not the determination condition C1 is satisfied (Step 201). Here, the line-of-sight determination section 60 measures duration T1 after it determines that the line-of-sight 3 (the viewpoint) of the speaker 1a has deviated from the character display area 10a, and the intention determination section 61 determines whether or not the duration T1 of a state that the line-of-sight 3 of the speaker is deviated is a predetermined threshold value or more.

If the duration T1 is greater than or equal to the threshold values (Yes in Step 201), it determines that the conveyance intention is absent (Step 202). If the duration T1 is less than the threshold values (No in Step 201), it determines that the conveyance intention is present (Step 203).

Thus, if the state that the line-of-sight 3 of the speaker 1a is deviated from the character display area 10a where the character information 5 is displayed continues for the certain period of time, it determines that the conveyance intention is absent. As a result, for example, it becomes possible to easily identify a case in which the speaker 1a temporarily confirms the facial expression or the like of the receiver 1b and a case in which the speaker 1a has no conveyance intention.

In FIG. 8, the determination process of the conveyance intention is executed based on the speech speed of the speaker 1a. In this determination process, a condition (hereinafter, referred to as a determination condition C2) indicating whether or not the speech speed of the speaker 1a exceeds a certain value as compared with a normal average value is determined based on the speech speed of the speaker 1a. The normal speech speed of the speaker 1a is learned in advance and stored in the storage section 52.

For example, if the speaker 1a is occupied by speaking, the speech speed of the speaker 1a often becomes quick. If the character information 5 is checked, the speaker 1a may utter more slowly. That is, the determination condition C2 can be said to be a condition for determining a condition in which the speaker 1a is occupied by speaking based on the speech speed.

The average value of the previous speech speed of speaker 1a is read from the storage section 52 (Step 301).

Next, it determines whether or not the determination condition C2 is satisfied (Step 302). Here, a difference obtained by subtracting the average value of the previous speech speed from the speech speed of the speaker 1a after the process of making the field of view of the speaker 1a difficult to look (a presentation process that it is difficult to look) is calculated, and it determines whether or not the difference of the speech speed is equal to or greater than the threshold value.

If the difference in the speech speed is greater than or equal to the threshold value (Yes in Step 302), it determines that the speech speed of the current speaker 1a is sufficiently fast and that the conveyance intention is absent (Step 303). If the difference in the speech speed is less than the threshold value (No in Step 302), it determines that the conveyance intention is present (Step 304).

As a result, for example, the state that the speaker 1a is occupied by speaking can be easily detected as the state that the conveyance intention is absent.

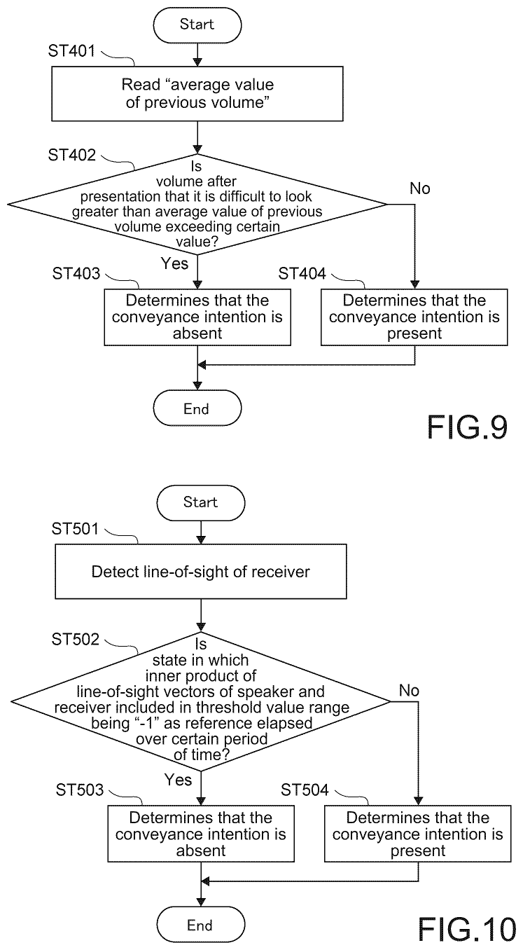

In FIG. 9, the determination process of the conveyance intention is executed based on the volume of the speaker 1a. In this determination process, a condition (hereinafter, referred to as a determination condition C3) indicating whether or not the volume of the speaker 1a exceeds a certain value as compared with a normal average value is determined based on the volume of the speaker 1a. The normal volume of the speaker 1a is learned in advance and stored in the storage section 52.

As in the case of the speech speed, if the speaker 1a is occupied by speaking, the volume of the speaker 1a often increases. That is, the determination state C3 can be said to be a condition for determining a condition in which the speaker 1a is occupied by speaking based on the volume.

The average value of the previous volume of the speaker 1a is read from the storage section 52 (Step 401).

Next, it determines whether or not the determination criterion C3 is satisfied (Step 402). Here, a difference obtained by subtracting the average value of the previous volume from the volume of the speaker 1a after the process of making the field of view of the speaker 1a difficult to look (the presentation process that it is difficult to look) is started is calculated, and it determines whether or not the difference of the volume is equal to or greater than a predetermined threshold value.

If the difference in the volume is greater than or equal to the threshold value (Yes in Step 402), it determines that the conveyance intention is absent, assuming that the volume of the current speaker 1a is sufficiently large (Step 403). If the difference in the volume is less than the threshold value (No in Step 402), it determines that the conveyance intention is present (Step 404).

As a result, for example, the state that the speaker 1a is occupied by speaking can be easily detected as the state that the conveyance intention is absent.

Note that as the determination condition regarding the speech speed and the volume, for example, the duration of a state that the speech speed and the volume exceed the threshold value, and the like may be determined. That is, it may determine whether or not the state that the difference in the speech speed and the difference in the volume are equal to or more than the threshold value continues for a predetermined time or more. As a result, it is possible to detect the state that the user is occupied by speaking with high accuracy.

In FIG. 10, the determination process of the conveyance intention is executed based on the line-of-sight 3 of the speaker 1a and the line-of-sight 3 of the receiver 1b. In this determination process, a condition (hereinafter, referred to as a determination condition C4) that a certain period of time elapses while the line-of-sight vector of the speaker 1a and the line-of-sight vector of the receiver 1b face each other is determined. More specifically, the state that the respective line-of-sight vectors of the speaker 1a and the receiver 1b face each other is a state that the inner product of the respective line-of-sight vectors is −1(=cos(180 degrees) as a reference when the respective line-of-sight vectors are set as unit vectors. This indicates that the speaker 1a and the receiver 1b look each other eye to eye.

For example, if the speaker 1a and the receiver 1b communicate with each other by looking each other eye to eye, it may be possible to forget that the communication is performed using the character information 5. The determination state C4 can be said to be a condition for determining such a condition based on the line-of-sights of the speaker 1a and the receiver 1b.

First, the line-of-sight 3 of the receiver 1b is detected (Step 501). For example, the line-of-sight 3 of the receiver 1b is estimated by the line-of-sight detection section 58 from the image of the receiver 1b captured by the face recognition camera 28a.

Alternatively, the line-of-sight 3 of the receiver 1b may be estimated based on the image of the eyeballs of the receiver 1b captured by the smart glass 20b (the line-of-sight detection camera 27b).

Next, it determines whether or not the determination criterion C4 is satisfied (Step 502). Here, an inner product value of the line-of-sight vector of the speaker 1a and the line-of-sight vector of the receiver 1b is calculated, and it determines whether or not the inner product value is included in a threshold value range being −1 as the minimum value. If the inner product value is included in the threshold value range, duration T2 is measured. Then, it determines whether or not the duration T2 is greater than or equal to a predetermined threshold value.

If the duration T2 is greater than or equal to the threshold value (Yes in Step 502), it determines that the speaker 1a and the receiver 1b are focused on looking and communicating with each other eye to eye and that the conveyance intention is absent (Step 503). If the duration T2 is less than the threshold value (No in Step 502), it determines that the conveyance intention is present (Step 504).

As a result, for example, a state that the speaker 1a is occupied by speaking with the eyes of the receiver 1b can be detected as the state that the conveyance intention is absent.

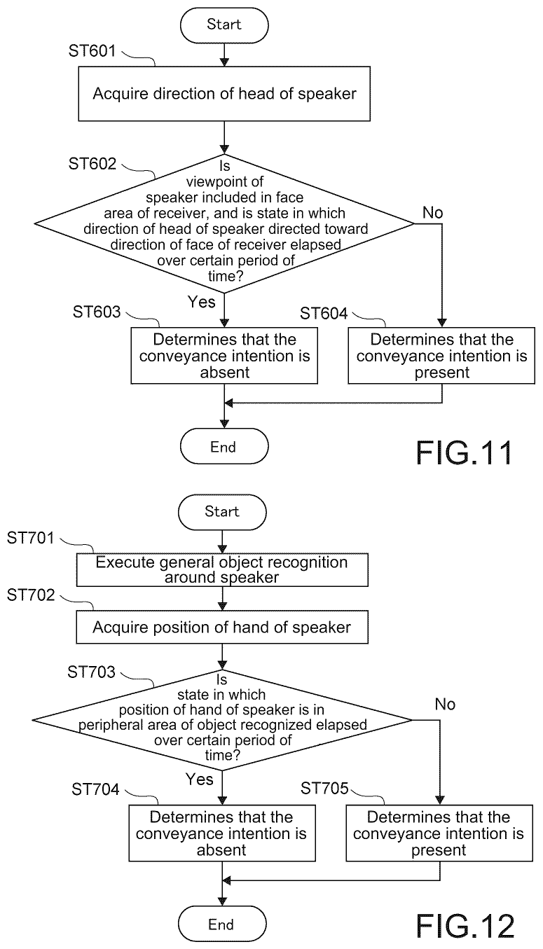

In FIG. 11, the determination process of the conveyance intention is executed based on the direction of the head of the speaker 1a. In this determination process, a condition (hereinafter, referred to as determination condition C5) that a certain period of time elapses while the direction of the head of the speaker 1 is directed toward the direction of the face of the receiver 1b while the line-of-sight 3 of the speaker 1a is directed toward the face of the receiver 1b is determined.

The determination condition C5 indicates a state that the line-of-sight 3 and the direction of the head of the speaker 1a are both directed toward the receiver 1b, that is, a state that the speaker 1a is concentrated on the face of the receiver 1b. As described above, if the user concentrates only on the facial expressions of the receiver 1b or the like, it is conceivable that the user forgets to communicate using the character information 5. The determination state C5 can also be said to be a condition for determining such a condition from the line-of-sight 3 and the direction of the head of the speaker 1a.

First, the direction of the head of the speaker 1a is acquired (Step 601). For example, the direction of the head of the speaker 1a is estimated based on the output of the acceleration sensor 29a mounted on the smart glass 20a.

Next, it determines whether or not the determination criterion C5 is satisfied (Step 602). Here, it determines, in the display screen 6a, whether or not the viewpoint of the speaker 1a is included in the face area of the receiver 1b (whether or not the speaker 1a looks at the face of the receiver 1b). Also, it determines whether or not the direction of the head of the speaker 1a faces the direction of the face of the receiver 1b. If these two determinations are Yes, then duration T3 of the state is measured. It is then determined whether or not the duration T3 is greater than or equal to a predetermined threshold value.

If the duration T3 is greater than or equal to the threshold value (Yes in Step 602), it determines that the speaker 1a is concentrated on the face of the receiver 1b and that the conveyance intention is absent (Step 603). If the duration T3 is less than the threshold value (No in Step 602), it determines that the conveyance intention is present (Step 604).

Thus, for example, a state that the speaker 1a is concentrated on the facial expression of the receiver 1b or the like can be detected as the state that the conveyance intention is absent.

In FIG. 12, based on the position of the hand of the speaker 1a, the determination process of the conveyance intention is executed. In this determination process, a condition (hereinafter, referred to as a determination condition C6) that a certain period of time elapses while the speaker 1a continues to operate the object around the speaker 1a with the hand is determined. Here, the object around the speaker 1a is, for example, a real object such as a document or a portable terminal. Alternatively, a virtual object or the like presented by the smart glass 20a is also included in an operation target of the speaker 1a.

For example, if the speaker 1a is operating a surrounding object (e.g., turning a document required for a meeting, operating a smartphone screen, etc.), there is a possibility that the speaker 1a concentrates on the operation and does not pay attention to the character information 5. The determination state C4 can be said to be a condition for determining such a condition based on the position of the hand of the speaker 1a.

First, general object recognition is executed on a space around the speaker 1a (Step 701). In the general object recognition, a process of detecting an object such as a document, a mobile phone, a book, a desk, or a chair is executed. For example, by performing image segmentation or the like on the image captured by the face recognition camera 28a, the object appearing in the image is detected.

Next, the position of the hand of the speaker 1a is acquired (Step 702). For example, the position of a palm of the speaker 1a is estimated from the image captured by the face recognition camera 28a.

Next, it determines whether or not the determination condition C6 is satisfied (Step 703). Here, it determines whether or not the position of the hand of the speaker 1a is in a peripheral area of the object recognized by the general object recognition. The peripheral area is an area set for each object so as to surround the object. If the position of the hand of the speaker 1a is included in the peripheral area, it is highly likely that the speaker 1a is operating on the object. Here, duration T4 of the state that the position of the hand of the speaker 1a is included in the peripheral area is measured. Then, it determines whether or not the duration T4 is greater than or equal to a predetermined threshold value.

If the duration T4 is greater than or equal to the threshold value (Yes in Step 703), it determines that the speaker 1a is focused on operating the object and that the conveyance intention is absent (Step 704). If the duration T4 is less than the threshold value (No in Step 703), it determines that the conveyance intention is present (Step 705).

As a result, for example, the state that the speaker 1a is concentrated on operation of the surrounding object can be detected as the state that the conveyance intention is absent.

In addition, a specific method of the determination process of the conveyance intention is not limited. For example, a determination condition based on biometric information such as a pulse and a blood pressure of the speaker 1a may be determined. Alternatively, a determination condition may be configured based on dynamic information such as an operation frequency of the line-of-sight 3 and an operation frequency of the head.

In addition, in the above-described cases, if one of the determination conditions C1 to C6 is satisfied, the determination process is performed to determine that the conveyance intention is absent. For example, a final determination result may be calculated by combining a plurality of the determination conditions.

### [Presentation Process of Conveyance Intention]

FIG. 13 is schematic diagrams each illustrating an exemplary process of presenting to the speaker 1a that the conveyance intention is absent. An exemplary presentation process executed in Step 110 of FIG. 5 is schematically illustrated in “A” to “E” of FIG. 13. Here, it is assumed that each presentation process is performed while the display screen 6a that makes the entire field of view difficult to look is displayed as shown in “A” of FIG. 6. Note that each process illustrated in FIG. 13 can be executed regardless of the types of the processes that make the field of view difficult to look.

Each presentation process illustrated in “A” and “B” of FIG. 13 is a process of visually presenting, to the speaker 1a, that the speaker 1a has no own conveyance intention by using the display 30a (the display screen 6a) visually recognized by the speaker 1a. In this case, the display screen 6a is controlled based on visual data which is generated by the output-control section 63 and indicates that the conveyance intention is absent.

In “A” of FIG. 13, the entire screen of the display screen 6a is blinked. For example, a background of a warning color such as red is displayed to blink. Accordingly, it is possible to reliably present to the speaker 1a that the conveyance intention is absent.

In “B” of FIG. 13, an edge (an outer periphery) of the display screen 6a is lit with a predetermined warning color. As a result, the speaker 1a can determine that the conveyance intention is absent in a peripheral field of view, and it becomes possible to naturally present that the conveyance intention is absent to the speaker 1a.

Furthermore, for example, if the conveyance intention is absent, control may be performed by illuminating a light-emitting device such as an LED or the like provided so that the speaker 1a can be visually recognized.

The presentation process illustrated in “C” of FIG. 13 is a process of presenting, to the speaker 1a, that own conveyance intention is absent by using a haptic sense. For haptic presentation, the vibration presentation section 31a mounted on the smart glass 20a is used. In this case, the vibration presentation section 31a is controlled based on the vibration data indicating that the conveyance intention is absent generated by the output control section 63.

For example, the vibration presentation section 31a is mounted on a frame (a temple) of the smart glass 20a or the like, and a direct vibration is presented on the head of the speaker 1a.

Also, for example, other haptic device 14 worn by the speaker 1a or carried by the speaker 1a may be vibrated warningly. For example, a device such as a neck-band speaker used by hanging over the neck of the speaker 1a or a haptic vest worn on the body of the speaker 1a and presenting various haptics to various parts of the body may be vibrated. Furthermore, a mobile terminal such as the smartphone used by the speaker 1a may be vibrated.

By performing the warning using the vibration in this way, for example, it is possible to effectively present that the conveyance intention is absent to the speaker 1a who is occupied by speaking or other operation.

The presentation process illustrated in “D” FIG. 13 is a process of presenting, to the speaker 1a, that the speaker 1a has no own conveyance intention by using a sound such as a warning sound or a warning speech. For sound representation, the speaker 32a mounted on the smart glass 20a is used. In this case, the speaker 32a reproduces the sound data indicating that the conveyance intention is absent, which is generated by the output control section 63. Also, for example, sound may be reproduced using other audio device (such as a neckband speaker and the smartphone) worn by the speaker 1a or carried by the speaker 1a.

In the example shown in “D” of FIG. 13, a feedback sound of “beep” is reproduced as the warning sound. In addition, a speech synthesis sound that conveys warning content may be reproduced. In this case, the synthesized sound “Please look at the text” is reproduced using the technique of TTS (Text to Speech).

By performing the warning using the speech, for example, it is possible to effectively present that the conveyance intention is absent to the speaker 1a who is occupied by speaking or other operation.

The presentation process illustrated in “E” of FIG. 13 is a process of changing the position of the character information 5 (the character display area 10a) displayed to the speaker 1a and presenting that the conveyance intention is absent displayed to the speaker 1a. Specifically, if it determines that the conveyance intention is absent, the character information 5 is displayed so as to intersect the line-of-sight 3 of the speaker 1a in the display 30a used by the speaker 1a.

As shown in the left side of “E” of FIG. 13, if the speaker 1a takes eyes off from the character information 5 (the character display area 10a), the transparency of the entire screen is decreased (see “A” of FIG. 6). Even if the transparency becomes low and the face of the receiver 1b cannot be looked, it is assumed that the line-of-sight 3 of the speaker 1a does not return to the character information 5 as is and that the viewpoint of the speaker 1a remains at the position of the face on the receiver 1b. In this case, the character information 5 of subsequent utterance is displayed at the position of the viewpoint of the speaker 1a, that is, at the position intersecting the line-of-sight 3 of the speaker 1a.

As a result, if it determines that the conveyance intention is absent, it is possible to positively present the speaker 1a that the character information 5 is not paid attention and to make confirm the character information 5 itself. As a result, it becomes possible to guide the speaker 1a to communicate using the character information 5.

### [Operation of Communication System on Receiver Side]

FIG. 14 is a flow chart illustrating an exemplary operation of a receiver side of the communication system. The process illustrated in FIG. 14 is a process for controlling the operation of the smart glass 1b mainly used by the receiver 20b, and is repeatedly executed while the speaker 1a and the receiver 1b communicate with each other. This process is executed in parallel with the process illustrated in FIG. 5, for example. The operation of the communication system 100 with respect to the receiver 1b will be described below with reference to FIG. 14.

In the communication system 100, the output control section 63 executes a process of conveying the receiver 1b that the speaker 1a has the conveyance intention using the character information 5. That is, if it determines that the conveyance intention is present, it is presented to at least the receiver 1b that the conveyance intention is present in the speaker 1a. By this process, the receiver 1b can easily determine whether or not to pay attention to the character information 5 and whether or not to make a speech.

In the following, a process of conveying the receiver 1b that the speaker 1a has the conveyance intention by presenting the dummy information to the receiver 1b will be described.

First, the output control section 63 reads a determination result of the conveyance intention (Step 801). Specifically, the information about the presence or absence of the conveyance intention, which is the result of the determination process (see FIGS. 7 to 12) executed in Step 107 of FIG. 5, is read.

Next, it determines whether or not the conveyance intention is absent (Step 802). If it determines that the conveyance intention is present (No in Step 802), it determines whether or not there is presentation information regarding the speech recognition (Step 803).

Here, the presentation information regarding the speech recognition is information for presenting, to the receiver 1b, that the speech recognition for the speaker 1a is being performed. For example, information indicating a detection state of the speech (e.g., volume information or the like of the speech) and a recognition result of the speech recognition (the character information 5) serve as the presentation information.

In the smart glass 20b, the presentation information is presented to the receiver 1b. For example, it is possible to convey the receiver 1b that the speech is detected by displaying an indicator or the like that changes depending on the volume information. Furthermore, by presenting the character information 5, it is possible to convey the receiver 1b that the speech recognition is being performed. By looking the information, the receiver 1b can determine whether or not the speaker 1a is uttering.

For example, in a state that the speaker 1a is not uttering, if it determines that there is no presentation information regarding the speech recognition (No in Step 803), the dummy information to resemble the state that the speaker 1a utters is generated (Step 804).

Specifically, a dummy effect (dummy volume information or the like) or a dummy character string that the speaker 1a appears to be uttering is generated as the dummy information by the dummy information generation section 62 described with reference to FIG. 4.

Once the dummy information is generated, a display process using the dummy effect is executed in the display 30b (the display screen 6b) of the smart glass 20b (Step 805). After the dummy effect is displayed, the dummy character string is displayed on the display 30b (the display screen 6b) (Step 806). The dummy effect and the dummy character string will be described in detail with reference to FIG. 15.

Returning to Step 803, in the state that the speaker 1a is uttering, it determines that there is presentation information regarding the speech recognition (Yes in Step 803). In this case, instead of the dummy effect, a process of changing the indicator or the like depending on the actual volume is executed. In addition, the speech recognition process is executed, and the character information 5 that is its recognition result is displayed on the display 30b (the display screen 6b) (Step 806). In Step 806, both the dummy character string and the original character information 5 may be displayed.

As described above, in the present embodiment, the dummy information is displayed on the display 30b used by the receiver 1b until the character information 5 indicating the utterance content of the speaker 1a is acquired by the speech recognition during the period in which the output control section 63 determines that the conveyance intention is present.

The dummy information is displayed if the speaker 1a has the conveyance intention but there is no presentation information regarding the speech recognition. This corresponds to, for example, a case in which the speaker 1a makes a long utterance at a time and the speech recognition process does not catch up, or a case in which the speaker 1a is thinking about speaking and the utterance is interrupted. In such cases, it becomes possible to present the display screen 6b to the receiver 1b, as if the speaker 1a were uttering.

As a result, it is possible to make the speaker 1a appears to be uttering during the time period until the character information 5 indicating the original utterance content of the speaker 1a is displayed.

Returning to Step 802, if it determines that the conveyance intention is absent (Yes in Step 802), it determines whether or not there is the presentation information regarding the speech recognition (Step 807) as in Step 803.

If it determines that there is no presentation information regarding the speech recognition (No in Step 807), it returns to Step 801, and next loop process is started.

If it determines that there is the presentation information regarding the speech recognition (Yes in Step 807), a process of suppressing the presentation information is executed (Step 808).

Here, the process of suppressing the presentation information is a process of intentionally suppressing the presentation even if the volume information or the character information 5 presented to the receiver 1b exists. For example, a process of stopping the display of the character information 5, alert information notifying that the conveyance intention is absent, and the like are displayed. The process can be said to be a process that directly or indirectly conveys to the receiver 1b that the conveyance intention is absent in the speaker 1a.

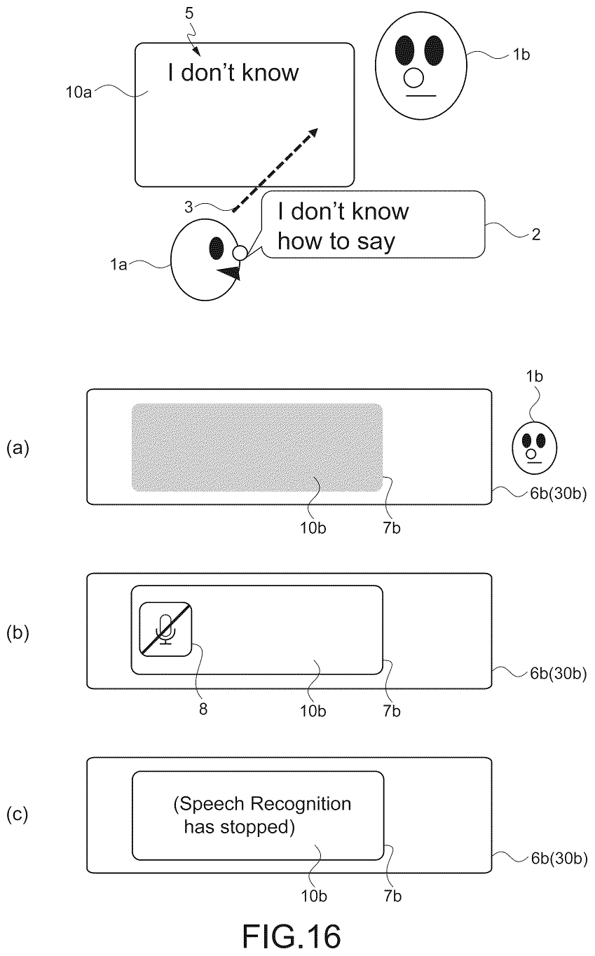

Once the suppression process of the presentation information is executed, it returns to Step 801, and the next loop process is started. The suppression process of the presentation information to the receiver 1b will be described in detail with reference to FIG. 16.

FIG. 15 is schematic diagrams each illustrating an exemplary process on the receiver 1b side if the conveyance intention is present. Here, an example in which the speaker 1a makes long utterance at a time will be described. As an example, the dummy information is displayed to the receiver 1b. An upper view of FIG. 15 schematically illustrates a display example of the display screen 6a (the display 30a of the smart glass 20a) of the speaker 1a side if the long utterance is made. In addition, (a) to (d) of FIG. 15 each schematically illustrates a display example of the dummy information on the display screen 6b (the display 30b of the smart glass 20b) of the receiver 1b side.

As shown in the upper view of FIG. 15, it is assumed that the speaker 1a utters a long sentence at a time. In this case, the speech recognition process takes time, and the recognition result (the character information 5) may not be displayed immediately after the utterance is completed. In particular, when the speaker 1a speaks quickly, it may not be possible even to show the intermediate result. As a consequence, the updating of the character information 5 stops as shown in the display screen 6a of FIG. 15. In addition, it is conceivable that a silent state occurs between the time when the utterance is completed and the time when the character information 5 is displayed.

At this time, since the receiver 1b cannot determine the presence or absence of the speech, it is difficult to determine whether or not the utterance is simply not made or the speech recognition is being processed.

Therefore, in the present embodiment, as described in Steps 804 to 806 of FIG. 14, if the speaker 1a has the conveyance intention, even when the recognition result of the speech recognition (the character information 5) and the volume information are not updated, the dummy information for resembling the state that the utterance is present is generated, and a supplementary presentation process is performed.

The presentation process of the dummy information is executed, for example, when the utterance of the speaker 1a ends and the volume is lost, and until the final result of the speech recognition process is returned, if there is no output of the character information 5 or no new audio input even after a certain period of time has elapsed from the last presentation of the character information 5.

(a) and (b) of FIG. 15 each shows the display example of the dummy information to be supplemental to the volume. This corresponds to the process of Step 805 of FIG. 14. Here, as the dummy information, information of the dummy effect that the speaker 1a appears to be uttering is used. The dummy effect information may be, for example, information specifying an effect or data for moving the effect. The dummy volume information generated using a random number or the like is used.

In (a) of FIG. 15, an indicator 15 that changes depending on the volume information is configured inside the microphone icon 8. If the speech of the speaker 1a is detected, the indicator 15 is displayed depending on the volume. Here, since the indicator 15 is displayed based on the dummy volume information in the state that the speech of the speaker 1a is not detected, it becomes possible that it appears as if a microphone volume were present.

In (b) of FIG. 15, the indicator 15 that changes depending on the volume information is configured at an edge (an outer peripheral part) of the display screen 6b. If the speech of the speaker 1a is detected, a color and brightness of the edge of the display screen 6b change depending on the volume. Since the display of the edge of the display screen 6b which becomes the indicator 15 is also changed based on the dummy volume information, it becomes possible that it appears as if the microphone volume were present.

(c) and (d) of FIG. 15 each shows a display example of the dummy information to be supplemental to the character information 5 that is the recognition result of the speech recognition. This corresponds to the process of Step 806 of FIG. 14. Here, as the dummy information, information of the dummy character string that makes the character information 5 look like output is used. The dummy character string may be, for example, a randomly generated character string or a fixed character string set in advance. Furthermore, for example, the dummy character string may be generated using a word or the like estimated from the content of the previous utterance or the like.

In (c) of FIG. 15, the randomly generated dummy character strings “XX”, “YY”, “LL”, “AA”, and the like are displayed following the character information “ABCD” that has already been outputted. This makes it possible to show a state that makes the speech recognition process appears to be continued.

In (d) of FIG. 15, the dummy character string “****” generated as the fixed character string is displayed following the character information “ABCD”. In this case, for example, by using a character or the like that is not a generating language, although the original character information 5 is not displayed, it can convey the receiver 1b that the speech recognition process is continuing.

The length of the dummy character string may be appropriately set based on, for example, an input time of the speech recognition (a length of the utterance).

FIG. 16 is schematic diagrams each illustrating an exemplary process on the receiver 1b side if the conveyance intention is absent. Here, an example will be described in which the speaker 1a self-talks, and the receiver 1b suppresses the presentation information regarding the speech recognition on the receiver 1b side. This process corresponds to the process of Step 808 of FIG. 14. In an upper view of FIG. 16, a display example of the display screen 6a on the speaker 1a side (the display 30a of the smart glass 20a) if the speaker 1a self-talks “I don't know how to say” is schematically illustrated. Furthermore, (a) to (c) of FIG. 16 each schematically illustrates the display screen 6b on the receiver 1b side (the display 30b of the smart glass 20b).

The self-talk of the speaker 1a is not the utterance that the speaker 1a tries to convey to the receiver 1b. Therefore, if the speaker 1a self-talks, the line-of-sight 3 is not directed to the character information 5, and it is considered that it determines that there is no conveyance intention. Under such situation, the receiver 1b does not need to pay attention to the character information 5, the facial expression of the speaker 1a, and the like.

As described above, if the speech recognition responds to the self-talk of the speaker 1a and displays as the character information 5, it takes a long time to prove it is the self-talk, and there is a possibility that an extra burden is imposed on the receiver 1b.

Therefore, in the present embodiment, as described in Step 808 of FIG. 14, if the speaker 1a has no conveyance intention, a process of suppressing displaying the presentation information (the character information 5, the volume information, and the like) regarding the speech recognition is performed.

In this process, for example, even when information to be presented/updated (such as the volume information and the character information 5 of the utterance of the speaker 1a) is acquired if the conveyance intention is present, the information is suppressed from being displayed if the conveyance intention is absent. This allows the receiver 1b to present that the speaker 1a has no conveyance intention by the character information 5.

In any of the processes illustrated in (a) to (c) of FIG. 16, the character information 5 is not displayed on the display screen 6b of the receiver 1b. That is, if it determines that the speaker 1a has no conveyance intention, the process of displaying the character information 5 in the display 30b (the display screen 6b) used by the receiver 1b is stopped. This eliminates the need for the receiver 1b to confirm the self-talk or to determine that the character information 5 is the self-talk.

Furthermore, when the process of displaying the character information 5 is stopped, the speech recognition process itself may be stopped.

(a) of FIG. 16 illustrates an example showing that, as in the case in which the speech recognition is OFF, the display of the character information 5 is deleted and the speech recognition is ended. For example, in a case in which the speech recognition is OFF, it is assumed that the background of the character display area 10b (the rectangular object 7b) for displaying the character information 5 is set to be gray. If it determines that the conveyance intention is absent, the character information 5 is deleted by making the background gray, for example, even if the speech recognition is actually operated.

(b) of FIG. 16 illustrates an example presenting that the display of the microphone icon 8 is changed and that the speech recognition is ended. Here, a diagonal line is added to the microphone icon 8. The display of the indicator 15 of the background of the microphone icon 8 is also stopped.

(c) of FIG. 16 illustrates an example presenting that a warning character is used to indicate that the speech recognition is ended. Here, the warning character in parentheses is displayed instead of the character information 5.

These processes can ensure that the speech recognition of the speaker 1a is not performed to the receiver 1b if the speaker 1a has no conveyance intention. As a consequence, the receiver 1b can recognize that it is not necessary to direct attention to the character information 5, the facial expression of the speaker 1a, and the like, and thus can open own vision.

As described above, in the controller 53 according to the present embodiment, the utterance of the speaker 1a is converted into characters by the speech recognition, and is displayed as the character information 5 on both the speaker 1a and the receiver 1b. At this time, based on the state of the speaker 1a, it determines whether or not the speaker 1a has the conveyance intention that the utterance content is conveyed to the receiver 1b using the character information 5, and the determination result is presented to the speaker 1a and the receiver 1b. As a result, for example, it becomes possible to encourage the speaker 1a to utter while confirming the character information 5, and to convey the receiver 1b that information such as whether or not to pay attention to the character information 5. As a result, smooth communication using the speech recognition can be realized.

In an application that supports the communication by displaying the result of the speech recognition, the utterance content of desired to be conveyed may not be successfully conveyed to the receiver depending on how the application is used by the speaker.

For example, when the speaker is occupied by speaking, the intention to “convey” what want to say “by the characters” may diminish, and the speaker may not look at the screen that displays the result of the speech recognition. In this case, even in a case in which a misrecognition occurs in the speech recognition, the speaker may continue to speak without being aware of the misrecognition, and the result of the misrecognition may continue to be conveyed to the receiver.

In addition, since the result of the speech recognition is continuously presented, it may be a burden on the receiver to keep consciousness of the result. In addition, in order to tell that “I don't understand” if the misrecognition or the like occurs, it is necessary to block the utterance of the speaker, and therefore it is difficult for the receiver to confirm the utterance content.

Moreover, in a constraint state that it is difficult to hear the speech, the presence or absence of the sound cannot be distinguished. Therefore, if the result of the speech recognition is not displayed, it is difficult for the receiver to distinguish whether or not there is no utterance or the result of the speech recognition is just not displayed. As a result, the receiver should continue to look the state of the mouth and the like of the speaker, which may increase the burden.

In addition, in many cases, a scene in which the speaker self-talks, a scene in which the speaker speaks toward the receiver, or the like cannot be distinguished only by the process of the speech recognition. As a result, once the speech recognition responds to the self-talk of the speaker, the receiver needs to wait until it proves that it is the self-talk, which results in wasted effort.

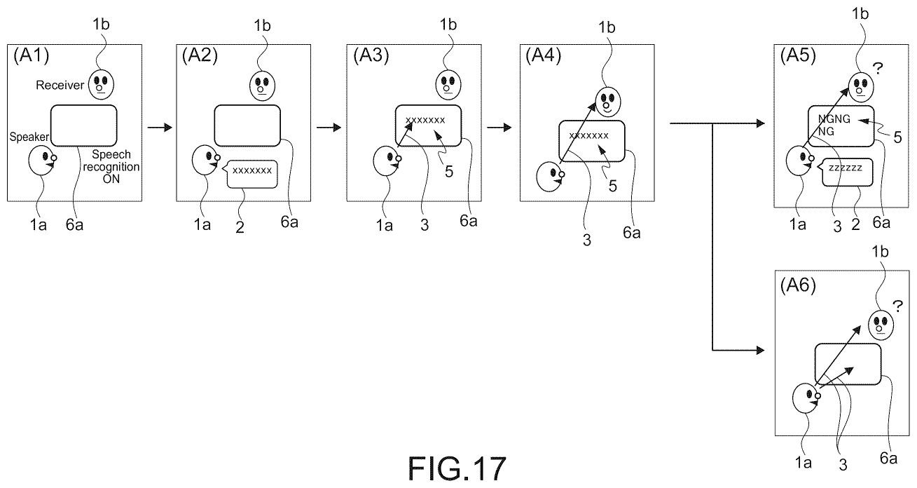

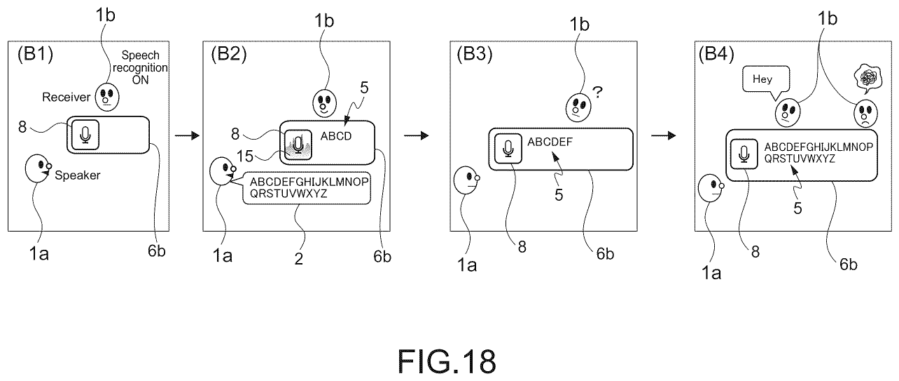

FIG. 17 and FIG. 18 are schematic diagrams each illustrating a display example of utterance character strings as a comparative example.

In FIG. 17, it determines that there is conveyance intention to convey the character information only when the speaker 1a deviates the line-of-sight 3 from the character information 5. In each step of (A1) to (A6) of FIG. 17, the display screen 6a of the speaker 1a side is illustrated.

First, the speech recognition is set to ON (A1) and the speech recognition for the speaker 1a is executed (A2). Next, the character information 5 that is the result of the speech recognition is displayed (A3). At this time, it determines whether or not the line-of-sight 3 of the speaker 1a is directed to the character information 5. It is assumed that the speaker 1a deviates the line-of-sight 3 from the character information 5 (A4).

In (A5), the speech recognition continues while the speaker 1a directs the line-of-sight 3 to the face of the receiver 1b. In this case, the speaker 1a may continue to look at the face of the receiver 1b and may not look at the screen. If there is no consciousness about the character information, it is not aware that the misrecognition or the like has occurred, and the receiver 1b cannot understand the meaning of the character information 5. In addition, the speaker 1a cannot easily recognize that the receiver 1b has not been understood.

In (A6), the speech recognition is set to OFF only by triggering that the line-of-sight 3 of the speaker 1a deviates from the character information 5. For example, in a case in which a conversation is performed, the line-of-sight 3 of the speaker 1a may frequently deviate from the character information 5a in order to frequently look the state and a response of the receiver 1b. For this reason, in the control in which the speech recognition becomes OFF every time the line-of-sight 3 is deviated from the character information 5, even if the speaker 1a thinks that the speaker 1a looks at the character information 5, the system determines that the character information 5 is not looked and the speech recognition is stopped. Therefore, the speech recognition is frequently stopped, and the character information 5 is not displayed as desired by the speaker 1a.

FIG. 18 schematically illustrates a case in which the speaker 1a makes long utterance at a time, and it takes a long time to display the result. In each of steps (B1) to (B4) of FIG. 18, the display screen 6b of the receiver 1b side is illustrated.

First, the speech recognition is set to ON (B1) and the speech recognition for the speaker 1a is started (B2). At this time, since the indicator 15 responds while the speaker 1a is uttering, the receiver 1b knows that the speaker 1a is uttering. Since the speaker 1a utters a large number of sentences at once, only a beginning of the utterance content in the character information 5 is displayed and the character information 5 will not be updated.

If the utterance of the speaker 1a is ended (B3), a speech process takes a long time, and the character information 5 is not updated. In this case, the operation appears to be stopped on the display screen 6b. The receiver 1b is aware that the character information 5 is not updated, but cannot hear the utterance, so it is difficult to determine whether or not the utterance is continuing.

Note that, since the speech recognition process continues even during a period in which the character information 5 is not updated, the character information 5 is finally displayed although there is a time lag.

Here, since the operation of the display screen 6b is stopped, it is assumed that the receiver 1b tries to talk to the speaker 1a. At this time, if the speaker 1a is uttering, the utterance may be blocked. For example, as shown in (B4), it is assumed that the receiver 1b performs an action to talk (here, say “Hey”). In such a case, if the character information 5 is suddenly updated, the action of the receiver 1b may be in vain or the communications may be rather hindered.

In addition, there is also a way to actively present that the speech recognition is in progress by a UI or the like, but there is a possibility that the receiver 1b or the speaker 1a may not be aware of such a display.

In the communication system 100 according to the present embodiment, it determines whether or not the speaker 1a tries to communicate by the conveyance intention using the speech-recognized character information 5, that is, the character information 5.

The determination result of the conveyance intention is presented to the speaker 1a oneself. Thus, if the speaker 1a concentrates on speaking and it determines that the character information 5 is not confirmed and the conveyance intention is absent, it becomes possible to encourage the speaker 1a to look at the character information 5.

As a result, it becomes possible that the speaker 1a can convey the content of the conversation to the receiver 1b when the speaker 1a speaks while confirming the recognition result of the speech recognition (the character information 5). In addition, the receiver 1b can receive the utterance content (the character information 5) spoken by the speaker 1a while confirming the content.

In addition, as to the utterance in the state that the conveyance intention is absent, the display of the character information 5 or the like is suppressed. As a result, the speaker 1a does not need to convey the speech recognition to the receiver 1b when the self-talk unintentionally occurs. The receiver 1b does not need to concentrate on the character information 5 or the like that is not needed to be checked.

As described above, when the speaker 1a confirms the own utterance content (the character information 5), as illustrated in (A5) of FIG. 17, for example, it is possible to avoid a situation in which the receiver 1b continues to look at the result of the misrecognition. In addition, since the speaker 1a can confirm a display amount or the like of the character information 5, it is possible to avoid a situation in which the character information is displayed unilaterally from the speaker 1a to the receiver 1b. Thus, the burden on the receiver 1b can be sufficiently reduced.

In addition, the determination result of the conveyance intention is presented to the receiver 1b. This allows the receiver 1b to easily determine whether or not the speaker 1a tries to communicate using the character information 5. Thus, for example, if the speaker 1a has no conveyance intention (see FIG. 16), the receiver 1b can easily determine that the speaker 1a does not try to convey the utterance to the receiver 1b. Therefore, the receiver 1b can immediately stop looking at the character information 5 or the facial expression of the speaker 1a (open the vision).

If the speaker 1a has the conveyance intention, the dummy information that appears as if there is the utterance of the speaker 1a or the operation of the speech recognition is displayed to the receiver 1b (see FIG. 15). Thus, the receiver 1b can easily determine whether or not the speaker 1a tries to continue the conversation.

In this way, the receiver 1b can intercept the conversation without hesitation when the speech recognition result is not attained. In addition, the receiver 1b can identify a waiting time until the character information 5 is displayed. Therefore, as shown in (B4) of FIG. 18, it can avoid a situation in which the character information 5 is displayed and the communication is disturbed when speaking to the speaker 1a during the waiting time.

Furthermore, in the present embodiment, if the line-of-sight 3 of the speaker 1a deviates from the character information 5 (the character display area 10a), the determination process of the conveyance intention is started. Therefore, even if the line-of-sight 3 deviates from the character information 5 as illustrated in (A6) of FIG. 17, it immediately stops the display of the character information 5, the speech recognition, or the like. Thus, for example, if the speaker 1a confirms the facial expression or the like of the receiver 1b, it becomes possible to avoid a situation in which speech recognition stops immediately. Thus, it becomes possible to provide an easy-to-use support system adapted to actual communication.

In addition, if the line-of-sight 3 of the speaker 1a deviates from the character information 5 (the character display area 10a), the process of making the field of view of the speaker 1a difficult to look (see FIG. 6 and the like) is executed. For example, even if a certain period of time is required to determine the conveyance intention, it is possible to warn the speaker 1a that the character information 5 has not been confirmed. In this way, by combining the process of making the field of view of the speaker 1a difficult to look, it becomes possible to warn the speaker 1a stepwise. As a result, the utterance of the speaker 1a is not hindered as little as possible, while an effective warning can be given if the conveyance intention is absent.

The speaker 1a can also create a situation that there is no conveyance intention on purpose. For example, when the speech recognition is not as intended by the speaker 1a, the speaker 1a can cancel the speech recognition by intentionally deviating the line-of-sight 3 from the character information 5. Furthermore, by returning the line-of-sight 3 to the character information 5 and starting the utterance again, it is possible to perform the speech recognition again.

In this way, the speaker 1a can communicate as desired by intentionally using the determination of the conveyance intention.

### OTHER EMBODIMENTS

The present technology is not limited to the embodiments described above, and can achieve various other embodiments.

In the above embodiments, the system using the smart glasses 20a and a 20b have been described. The type of the display device is not limited. For example, any display device applicable to technologies such as AR (Augmented Reality), VR (Virtual Reality) and MR (Mixed Reality) may be used. The smart glass is a spectacle-type HMD suitably used, for example, for the AR and the like. Alternatively, an immersive type HMD or the like configured to cover the head of the wearer may be used.

In addition, a portable device such as a smartphone or a tablet may be used as the display device. In this case, the speaker and the receiver communicate with each other via the character information displayed on the smartphone.

Furthermore, for example, a digital signage device that provides a digital outdoor advertisement (DOOH: Digital Out of Home), a user support service on a street, or the like may be used. In this case, communication is performed via the character information displayed on the signage device.

In addition, a transparent display, a PC monitor, a projector, a TV device, or the like can be used as the display device. For example, the utterance content of the speaker is displayed as the characters on the transparent display arranged at a window or the like. When performing remote video communication or the like, the display device such as the PC monitor may be used.

In the above-described embodiment, the case has been described in which the speaker and the receiver actually face each other to perform the communication. It is not limited thereto, and may be applied to the conversation at a remote meeting or the like. In this case, the character information obtained by converting the utterance of the speaker into the characters by the speech recognition is displayed on the PC screen and the like used by both the speaker and the receiver. In addition, if the speaker takes eyes off from the character information, a process of making the face or the like of the receiver difficult to look in the video of the receiver displayed on a speaker side, a process of displaying a warning at a line-of-sight position of the speaker, or the like is executed. On the receiver side, if the speaker has no conveyance intention, a process of stopping the display of the character information is executed.

Furthermore, the present technology is not limited to one-to-one communication between the speaker and the receiver, and can be applied to a case in which there are other participants. For example, in a case in which a hearing-impaired receiver speaks with a plurality of speakers who are healthy hearing persons, it determines the presence or absence of the conveyance intention by the character information for each speaker. It determines whether or not the utterance content is tried to be conveyed to the receiver for whom the character information is important. By applying the present technology to each speaker, the receiver can quickly know that even a plurality of conversations is not tried to be conveyed to receiver oneself, and it is not necessary to keep eyes on surrounding mouths to keep watch whether or not each speaker is speaking. This makes it possible to sufficiently reduce the burden on the receiver.

The present technology may be used for a translation conversation or the like in which the utterance content of the speaker is translated and conveyed to the receiver. In this case, the speech recognition is performed on the utterance of the speaker, and the recognized character string is translated. In addition, the character information before the translation is displayed to the speaker, and the translated character information is displayed to the receiver. Even in such a case, the presence or absence of the conveyance intention of the speaker is determined, and the determination result is presented to the speaker or the receiver. Accordingly, it is possible to avoid a situation in which the speaker is encouraged to utter while confirming the character information, or the translated sentence of the character string that has been misrecognized is continuously presented to the receiver.

It is also possible to use the present technology if the speaker makes a presentation. For example, in a case in which the character information (the character string of the utterance itself or the translated character string) indicating the utterance content at the time of presentation is displayed as a caption, the character information can be appropriately checked, so that the correction can be immediately performed even if the erroneous character string or the like is displayed.

In the above description, a process of presenting that the conveyance intention is present by displaying the dummy information to the receiver if the speaker has the conveyance intention has been described (see FIG. 15 and the like). For example, it may present to the speaker that the speaker oneself has the conveyance intention. For example, in a state that a conversation is performed while paying attention to the character information and it determines that the conveyance intention is present, the periphery of the screen may be turned on blue, and in a case in which it determines that the conveyance intention is absent, the periphery of the screen may be controlled to be turned on red. As a result, it is possible to convey the speaker that the speaking can be proceeded properly while the blue color is lit. As a result, it is possible to avoid a situation in which the speaker unnecessarily concentrates on the character information, and to realize natural communication.

As described in (A6) of FIG. 17, a process of stopping the speech recognition by using only that the line-of-sight of the speaker deviates from the character information may be executed. For example, in a case in which the speaker needs to concentrate sufficiently on the character information (such as an operation by conversation), it may determine the presence or absence of the conveyance intention under such a strong condition.

In the above description, the case in which the information processing method according to the present technology is executed by the computer of the system control section has been described. However, the information processing method and the program according to the present technology may be executed by the computer mounted in the system control section and another computer that can communicate with the computer via a network or the like.

That is, the information processing method and the program according to the present technology can be executed not only in a computer system configured by a single computer but also in a computer system in which a plurality of computers operates in conjunction with each other. Note that, in the present disclosure, the system refers to a set of plural components (such as apparatuses and modules (parts)) and it does not matter whether all of the components are in a single housing. Thus, a plurality of apparatuses accommodated in separate housings and connected to each other via the network, and a single apparatus in which a plurality of modules is accommodated in a single housing are both the system.

The execution of the information processing method and the program according to the present technology by the computer system include, for example, both cases in which a process of acquiring the character information of the speaker, a process of determining the presence or absence of the conveyance intention by the character information, a process of displaying the character information to the speaker or the receiver, and a process of presenting a determination result of the conveyance intention are executed by a single computer and are executed by different computers. Furthermore, the execution of each process by a specified computer includes causing another computer to execute a part of or all of the processes and acquiring results thereof.

In other words, the information processing method and the program according to the present technology are also applicable to a configuration of cloud computing in which a single function is shared and cooperatively processed by a plurality of apparatuses via the network.

It is also possible to combine at least two of the features of the present technology described above. In other words, various features described in the respective embodiments may be combined with no distinction among the embodiments. Furthermore, the various effects described above are not limitative but are merely illustrative, and other effects may be provided.

In the present disclosure, “same”, “equal”, “perpendicular”, and the like are concepts including “substantially the same”, “substantially equal”, “substantially perpendicular”, and the like. For example, states included in a predetermined range (e.g., range of +10%) with reference to “completely the same”, “completely equal”, “completely perpendicular”, and the like are also included.

Note that the present technology may also take the following configurations.

(1) An information processing apparatus, including
an acquisition section that acquires character information obtained by converting utterance of a speaker into characters by speech recognition;
a determination section that determines presence or absence of a conveyance intention that the speaker tries to convey speaker's own utterance content to a receiver using the character information based on a state of the speaker; and
a control section that executes a process of displaying the character information on display devices used by respective of the speaker and the receiver, and a process of presenting a determination result regarding the conveyance intention to at least one of the speaker and the receiver.
   (2) The information processing apparatus according to (1), in which
the control section generates notification data for notifying at least one of the speaker and the receiver that the conveyance intention is absent if it determines that the conveyance intention is absent.
   (3) The information processing apparatus according to (2), in which
the notification data includes at least one of visual data, haptic data, and sound data.
   (4) The information processing apparatus according to any one of (1) to (3), further including:
a line-of-sight detection section that detects a line-of-sight of the speaker; and
a line-of-sight determination section that determines whether or not the line-of-sight of the speaker is deviated from an area in which the character information is displayed in a display device used by the speaker based on a detection result of the line-of-sight of the speaker, in which
the determination section starts a determination process of the conveyance intention if the line-of-sight of the speaker deviates from the area in which the character information is displayed.
   (5) The information processing apparatus according to (4), in which
the determination section executes the determination process of the conveyance intention based on at least one of the line-of-sight of the speaker, a speech speed of the speaker, a volume of the speaker, a head direction of the speaker, or a hand position of the speaker.
   (6) The information processing apparatus according to (5), in which
the determination section determines that the conveyance intention is absent if a state that the line-of-sight of the speaker is deviated from the area in which the character information is displayed continues for a predetermined time.
   (7) The information processing apparatus according to (5) or (6), in which
the determination section executes the determination process of the conveyance intention based on the line-of-sight of the speaker and a line-of-sight of the receiver.
   (8) The information processing apparatus according to any one of (4) to (7), in which
the control section executes a process of making a field of view of the speaker difficult to look if the line-of-sight of the speaker deviates from the area in which the character information is displayed.
   (9) The information processing apparatus according to (8), in which
the control section sets a speed at which the field of view of the speaker is difficult to look based on at least one of reliability of the speech recognition, the speech speed of the speaker, a motion tendency of the line-of-sight of the speaker, or a noise level around the speaker.
   (10) The information processing apparatus according to (8) or (9), in which
the display device used by the speaker is a transmissive display device, and
the control section executes at least one of a process of decreasing transparency of at least a part of the transmissive display device or a process of displaying an object that blocks the field of view of the speaker on the transmissive display device, as the process of making the field of view of the speaker difficult to look.
   (11) The information processing apparatus according to any one of (8) to (10), in which
the control section cancels the process of making the field of view of the speaker difficult to look if the line-of-sight of the speaker returns to the area in which the character information is displayed.
   (12) The information processing apparatus according to any one of (1) to (11), in which
the control section displays the character information so as to intersect the line-of-sight of the speaker in the display device used by the speaker if it determines that the conveyance intention is absent.
   (13) The information processing apparatus according to any one of (1) to (12), in which
the control section executes a suppression process regarding the speech recognition if it determines that the conveyance intention is absent.
   (14) The information processing apparatus according to (13), in which
the control section stops a speech recognition process or stops the process of displaying the character information on at least one of the display devices used by the respective of the speaker and the receiver, as the suppression process.
   (15) The information processing apparatus according to any one of (1) to (14), in which
the control section presents to at least the receiver that the conveyance intention is present if it determines that the conveyance intention is present.
   (16) The information processing apparatus according to (15), further including:
a dummy information generation section that generates dummy information that the speaker appears to be uttering even if a speech of the speaker is absent, in which
the control section displays the dummy information on the display device used by the receiver until the character information indicating the utterance content of the speaker is acquired by the speech recognition during a period in which it determines that the conveyance intention is present.
   (17) The information processing apparatus according to (16), in which
the dummy information includes at least one of information of a dummy effect that the speaker appears to be uttering or information of a dummy character string that the character information appears to be outputted.
   (18) An information processing method executed by a computer system, including:
acquiring character information obtained by converting utterance of a speaker into characters by speech recognition;
determining presence or absence of a conveyance intention that the speaker tries to convey speaker's own utterance content to a receiver using the character information based on a state of the speaker;
executing a process of displaying the character information on display devices used by respective of the speaker and the receiver; and
executing a process of presenting a determination result regarding the conveyance intention to at least one of the speaker and the receiver.
   (19) A program that causes a computer system to execute:
a step of acquiring character information obtained by converting utterance of a speaker into characters by speech recognition;
a step of determining presence or absence of a conveyance intention that the speaker tries to convey speaker's own utterance content to a receiver using the character information based on a state of the speaker;
a step of executing a process of displaying the character information on display devices used by respective of the speaker and the receiver; and
a step of executing a process of presenting a determination result regarding the conveyance intention to at least one of the speaker and the receiver.

### REFERENCE SIGNS LIST

1a speaker
1b receiver
2 speech
3 line-of-sight
5 character information
6a, 6b display screen
10a, 10b character display area
20, 20a, 20b smart glass
30a, 30b display
50 system control section
51 communication section
52 storage section
53 controller
57 face recognition section
58 line-of-sight detection section
59 speech recognition section
60 line-of-sight determination section
61 intention determination section
62 dummy information generation section
63 output control section
100 communication system

1. An information processing apparatus, comprising:
an acquisition section that acquires character information obtained by converting utterance of a speaker into characters by speech recognition;
a determination section that determines presence or absence of a conveyance intention that the speaker tries to convey speaker's own utterance content to a receiver using the character information based on a state of the speaker; and
a control section that executes a process of displaying the character information on display devices used by respective of the speaker and the receiver, and a process of presenting a determination result regarding the conveyance intention to at least one of the speaker and the receiver.

2. The information processing apparatus according to claim 1, wherein
the control section generates notification data for notifying at least one of the speaker and the receiver that the conveyance intention is absent if it determines that the conveyance intention is absent.

3. The information processing apparatus according to claim 2, wherein
the notification data includes at least one of visual data, haptic data, and sound data.

4. The information processing apparatus according to claim 1, further comprising:
a line-of-sight detection section that detects a line-of-sight of the speaker; and
a line-of-sight determination section that determines whether or not the line-of-sight of the speaker is deviated from an area in which the character information is displayed in the display device used by the speaker based on a detection result of the line-of-sight of the speaker, wherein
the determination section starts a determination process of the conveyance intention if the line-of-sight of the speaker deviates from the area in which the character information is displayed.

5. The information processing apparatus according to claim 4, wherein
the determination section executes the determination process of the conveyance intention based on at least one of the line-of-sight of the speaker, a speech speed of the speaker, a volume of the speaker, a head direction of the speaker, or a hand position of the speaker.

6. The information processing apparatus according to claim 5, wherein
the determination section determines that the conveyance intention is absent if a state that the line-of-sight of the speaker is deviated from the area in which the character information is displayed continues for a predetermined time.

7. The information processing apparatus according to claim 5, wherein
the determination section executes the determination process of the conveyance intention based on the line-of-sight of the speaker and a line-of-sight of the receiver.

8. The information processing apparatus according to claim 4, wherein
the control section executes a process of making a field of view of the speaker difficult to look if the line-of-sight of the speaker deviates from the area in which the character information is displayed.

9. The information processing apparatus according to claim 8, wherein
the control section sets a speed at which the field of view of the speaker is difficult to look based on at least one of reliability of the speech recognition, the speech speed of the speaker, a motion tendency of the line-of-sight of the speaker, or a noise level around the speaker.

10. The information processing apparatus according to claim 8, wherein
the display device used by the speaker is a transmissive display device, and
the control section executes at least one of a process of decreasing transparency of at least a part of the transmissive display device or a process of displaying an object that blocks the field of view of the speaker on the transmissive display device, as the process of making the field of view of the speaker difficult to look.

11. The information processing apparatus according to claim 8, wherein
the control section cancels the process of making the field of view of the speaker difficult to look if the line-of-sight of the speaker returns to the area in which the character information is displayed.

12. The information processing apparatus according to claim 1, wherein
the control section displays the character information so as to intersect the line-of-sight of the speaker in the display device used by the speaker if it determines that the conveyance intention is absent.

13. The information processing apparatus according to claim 1, wherein
the control section executes a suppression process regarding the speech recognition if it determines that the conveyance intention is absent.

14. The information processing apparatus according to claim 13, wherein
the control section stops a speech recognition process or stops the process of displaying the character information on at least one of the display devices used by the respective of the speaker and the receiver, as the suppression process.

15. The information processing apparatus according to claim 1, wherein
the control section presents to at least the receiver that the conveyance intention is present if it determines that the conveyance intention is present.

16. The information processing apparatus according to claim 15, further comprising:
a dummy information generation section that generates dummy information that the speaker appears to be uttering even if a speech of the speaker is absent, wherein
the control section displays the dummy information on the display device used by the receiver until the character information indicating the utterance content of the speaker is acquired by the speech recognition during a period in which it determines that the conveyance intention is present.

17. The information processing apparatus according to claim 16, wherein
the dummy information includes at least one of information of a dummy effect that the speaker appears to be uttering or information of a dummy character string that the character information appears to be outputted.

18. An information processing method executed by a computer system, comprising:
acquiring character information obtained by converting utterance of a speaker into characters by speech recognition;
determining presence or absence of a conveyance intention that the speaker tries to convey speaker's own utterance content to a receiver using the character information based on a state of the speaker;
executing a process of displaying the character information on display devices used by respective of the speaker and the receiver; and
executing a process of presenting a determination result regarding the conveyance intention to at least one of the speaker and the receiver.

19. A program that causes a computer system to execute:
a step of acquiring character information obtained by converting utterance of a speaker into characters by speech recognition;
a step of determining presence or absence of a conveyance intention that the speaker tries to convey speaker's own utterance content to a receiver using the character information based on a state of the speaker;
a step of executing a process of displaying the character information on display devices used by respective of the speaker and the receiver; and
a step of executing a process of presenting a determination result regarding the conveyance intention to at least one of the speaker and the receiver.
